(19) United States
(12) Patent Application Publication
(10) Pub. No.: US 2026/0078188 A1
(43) Pub. Date: Mar. 19, 2026

(54) METHODS OF TREATING GRAVES’ DISEASE USING ANTI-FCRN ANTIBODIES

(71) Applicant: Immunovant Sciences GmbH, Basel (CH)

(72) Inventors: Peter Salzmann, New York, NY (US); Lauren Schrier, Tenafly, NJ (US); Philip Tedeschi, Gladstone, NJ (US); William Macias, Indianapolis, IN (US)

(73) Assignee: Immunovant Sciences GmbH, Basel (CH)

(21) Appl. No.: 19/109,035

(22) PCT Filed: Sep. 5, 2023
(86) PCT No.: PCT/EP2023/074345

§ 371 Date: Mar. 5, 2025

Related U.S. Application Data
(60) Provisional application No. 63/374,649, filed on Sep. 6, 2022.
(60) Provisional application No. 63/500,233, filed on May 4, 2023.

(51) Int. Cl. C07K 16/28

(52) U.S. Cl. CPC C07K 16/283; C07K 2317/21

(57) ABSTRACT

The present disclosure relates to methods of treating Graves' Disease using anti-FcRn antibodies.

### CROSS-REFERENCE TO RELATED APPLICATIONS

This application claims the benefit of U.S. Provisional Patent Application No. 63/374,649, filed Sep. 6, 2023, and U.S. Provisional Patent Application No. 63/500,233, filed May 4, 2023, each of which is incorporated by reference herein in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “MUNO-010_01WO_ST26”. The XML file is 66,026 bytes in size, created on Aug. 18, 2023, and is being submitted electronically via USPTO Patent Center.

### FIELD

The present disclosure relates to methods of treating Graves' Disease using anti-FcRn antibodies.

### BACKGROUND

Graves' Disease is an autoimmune disorder that can cause hyperthyroidism. The time from emergence of symptoms to seeking treatment is often more than 3 months and ˜40% of patients may receive a misdiagnosis (Stern et al., J Neuropsychiatry Clin Neurosci. 1996 Spring; 8(2):181-5). The time from seeking treatment to Graves' Diagnosis is often 2.5 months (Stern et al.). Standard of care for Graves' Disease leaves many difficult to control patients with poor quality of life and may also require thyroid ablation that is associated with long term challenges. The poor quality of life primarily is driven by elevated thyroid hormone levels that are a direct result of overstimulation of the thyroid gland by anti-TSHr auto-antibodies. ¼ to ⅓ of the 116,000 US incident Graves' patients are difficult to control with anti-thyroid medications and remain symptomatic (Zimmermann et al., Lancet Diabetes Endocrinol. 2015 April; 3(4):286-95; Furszyfer at al., Mayo Clin Proc. 1970 September; 45(9):636-44). Therefore, there remains an unmet need for effective and well-tolerated treatments for Graves' Disease.

Antibodies are immunological proteins that bind to a specific antigen. In most animals, including humans and mice, antibodies are constructed from paired heavy and light polypeptide chains and each chain is made up of two distinct regions, referred to as the variable and constant regions. The heavy and light chain variable regions show significant sequence diversity between antibodies and are responsible for binding to the target antigen. The constant regions show less sequence diversity and are responsible for binding a number of natural proteins to elicit various biochemical events.

Under normal conditions, the average serum half-life of most IgG (i.e., IgG1, IgG2, and IgG4, excluding the IgG3 isotype) is about 21 days in humans (Morell et al., J. Clin. Invest. 49(4):673-80, 1970), which is a prolonged period relative to the serum half-life of other plasma proteins. With respect to this prolonged serum half-life of IgG, IgG that enters cells by endocytosis can strongly bind to neonatal Fc receptor (FcRn) in endosomes at a pH of 6.0 to avoid the degradative lysosomal pathway (FcRn, a type of Fc gamma receptor, is also referred to as FcRP, FcRB, or Brambell receptor). When the IgG-FcRn complex cycles to the plasma membrane, IgG dissociates rapidly from FcRn in the bloodstream at slightly basic pH (˜7.4). By this receptor-mediated recycling mechanism, FcRn effectively rescues the IgG from degradation in lysosomes, thereby prolonging the half-life of IgG (Roopenian et al., J. Immunol. 170:3528, 2003).

FcRn is a non-covalent heterodimer that typically resides in the endosomes of endothelial and epithelial cells. FcRn is a membrane bound receptor having three heavy chain alpha domains (α1, α2, and α3) and a single soluble light chain β2-microglobulin (β2m) domain. Structurally, it belongs to a family of major histocompatibility complex class 1 molecules that have β2m as a common light chain. The FcRn chain has a molecular weight of about 46 kDa and is composed of an ectodomain containing the α1, α2, and α3 heavy chain domains and a β2m light chain domain and having a single sugar chain, a single-pass transmembrane, and a relatively short cytoplasmic tail.

### SUMMARY

Provided herein is a method of treating Graves' Disease in a patient in need thereof, comprising administering to the patient a first therapeutically effective amount of an anti-FcRn antibody or an antigen-binding fragment thereof, wherein the antibody or antigen-binding fragment comprises (a) a heavy chain variable region comprising a HCDR1 comprising the amino acid sequence set forth in SEQ ID No: 27, a HCDR2 comprising the amino acid sequence set forth in SEQ ID No: 28, and a HCDR3 comprising the amino acid sequence set forth in SEQ ID No: 29; and a light chain variable region comprising an LCDR1 comprising the amino acid sequence set forth in SEQ ID No: 30, an LCDR2 comprising the amino acid sequence of SEQ ID No: 31, and an LCDR3 comprising the amino acid sequence set forth in SEQ ID No: 32; or (b) a heavy chain variable region comprising a HCDR1 comprising the amino acid sequence set forth in SEQ ID No: 49, a HCDR2 comprising the amino acid sequence set forth in SEQ ID No: 22, and a HCDR3 comprising the amino acid sequence set forth in SEQ ID No: 23; and a light chain variable region comprising an LCDR1 comprising the amino acid sequence set forth in SEQ ID No: 50, an LCDR2 comprising the amino acid sequence of SEQ ID No: 25, and an LCDR3 comprising the amino acid sequence set forth in SEQ ID No: 26.

In some embodiments, the antibody or antigen-binding fragment comprises (a) a heavy chain variable region comprising an amino acid sequence that is at least 90%, at least 95%, at least 98%, or least 99% identical to the sequence set forth in SEQ ID No: 6, and a light chain variable region comprising an amino acid sequence that is at least 90%, at least 95%, at least 98%, or least 99% identical to the sequence set forth in SEQ ID NO: 16; or (b) a heavy chain variable region comprising an amino acid sequence that is at least 90%, at least 95%, at least 98%, or least 99% identical to the sequence set forth in SEQ ID No: 51, and a light chain variable region comprising an amino acid sequence that is at least 90%, at least 95%, at least 98%, or least 99% identical to the sequence set forth in SEQ ID NO: 52. In some embodiments, the antibody or antigen-binding fragment comprises (a) a heavy chain variable region comprising the sequence set forth in SEQ ID No: 6, and a light chain variable region comprising the sequence set forth in SEQ ID NO: 16; or (b) a heavy chain variable region comprising the sequence set forth in SEQ ID No: 51, and a light chain variable region comprising the sequence set forth in SEQ ID NO: 52.

In some embodiments, the antibody or antigen-binding fragment binds to FcRn with a K_{D} (dissociation constant) of 0.01 nM to 2 nM at pH 6.0 or pH 7.4. In some embodiments, the K_{D} is measured by surface plasmon resonance (SPR).

In some embodiments, the antibody or antigen-binding fragment is administered subcutaneously. In some embodiments, the antibody or antigen-binding fragment is administered once weekly.

In some embodiments, the first therapeutically effective amount of the antibody or antigen-binding fragment is 500 mg to 700 mg. In some embodiments, the first therapeutically effective amount of the antibody or antigen-binding fragment is 680 mg.

In some embodiments, the first effective amount of the antibody or antigen-binding fragment is administered for 12 weeks.

In some embodiments, the method further comprises administering a second therapeutically effective amount of the antibody or antigen-binding fragment to the subject. In some embodiments, the second therapeutically effective amount is 340 mg. In some embodiments, the second therapeutically effective amount of the antibody or antigen-binding fragment is administered for 12 weeks.

In some embodiments, the antibody, antigen-binding fragment, or pharmaceutical composition is administered in combination with at least one additional therapeutic agent.

In some embodiments, the patient is receiving one or more antithyroid drug (ATD). In some embodiments, the amount of ATD the patient is receiving is reduced after administration of the first or second effective amount of the antibody or antigen-binding fragment thereof.

In another aspect, provided herein is a method of treating Graves' Disease in a patient in need thereof, comprising administering to the patient a first therapeutically effective amount of an anti-FcRn antibody or an antigen-binding fragment thereof, wherein the antibody or antigen-binding fragment comprises: (a) a heavy chain variable region comprising a HCDR1 comprising the amino acid sequence set forth in SEQ ID No: 27, a HCDR2 comprising the amino acid sequence set forth in SEQ ID No: 28, and a HCDR3 comprising the amino acid sequence set forth in SEQ ID No: 29; and a light chain variable region comprising an LCDR1 comprising the amino acid sequence set forth in SEQ ID No: 30, an LCDR2 comprising the amino acid sequence of SEQ ID No: 31, and an LCDR3 comprising the amino acid sequence set forth in SEQ ID No: 32; or (b) a heavy chain variable region comprising a HCDR1 comprising the amino acid sequence set forth in SEQ ID No: 49, a HCDR2 comprising the amino acid sequence set forth in SEQ ID No: 22, and a HCDR3 comprising the amino acid sequence set forth in SEQ ID No: 23; and a light chain variable region comprising an LCDR1 comprising the amino acid sequence set forth in SEQ ID No: 50, an LCDR2 comprising the amino acid sequence of SEQ ID No: 25, and an LCDR3 comprising the amino acid sequence set forth in SEQ ID No: 26; and wherein the first therapeutically effective amount of the antibody or antigen-binding fragment is 680 mg administered subcutaneously once weekly, optionally wherein the administering is for about 12 weeks or more.

In some embodiments, the method further comprises administering a second therapeutically effective amount of the antibody or antigen-binding fragment to the patient, wherein the second therapeutically effective amount is 340 mg administered subcutaneously once weekly, optionally wherein the administering is for about 12 weeks or more.

In some embodiments, the patient is human.

### BRIEF DESCRIPTION OF DRAWINGS

FIG. 1 is a schematic showing a Phase 2 clinical trial of anti-FcRn antibodies in Graves' Disease. A: Additional inclusion and exclusion criteria not listed. GD=Graves' Disease; ATD=anti-thyroid medications; QW=weekly; SC=subcutaneous injection.

### DETAILED DESCRIPTION

In order that the disclosure may be more readily understood, certain terms are defined throughout the detailed description. Unless defined otherwise herein, all scientific and technical terms used in connection with the present disclosure have the same meaning as commonly understood by those of ordinary skill in the art. All references cited herein are also incorporated by reference for any purpose. To the extent a cited reference conflicts with the disclosure herein, the specification will control.

As used herein, the singular forms of a word also include the plural form, unless the context clearly dictates otherwise; as examples, the terms “a,” “an,” and “the” are understood to be singular or plural. By way of example, “an element” means one or more element. The term “or” means “and/or” unless the specific context indicates otherwise. All ranges, including those stated in the form of “between value X and value Y,” include the endpoints and all points in between unless the specific context indicates otherwise.

### Methods of Treatment

In some embodiments, the present disclosure is directed to a method of treating Graves' Disease by administering an anti-FcRn antibody or an antigen-binding fragment thereof, or by administering a pharmaceutical composition comprising at least one pharmaceutically acceptable carrier and an anti-FcRn antibody or an antigen-binding fragment thereof, to a patient in need of treatment. In some embodiments, the present disclosure is directed to a use of an anti-FcRn antibody or an antigen-binding fragment thereof in a method of treating Graves' Disease by administering the anti-FcRn antibody or antigen-binding fragment, or by administering a pharmaceutical composition comprising at least one pharmaceutically acceptable carrier and the anti-FcRn antibody or antigen-binding fragment, to a patient in need of treatment. In some embodiments, the present disclosure is directed to a use of an anti-FcRn antibody or an antigen-binding fragment thereof in the manufacture of a medicament for treating Graves' Disease. In some embodiments, the present disclosure is directed to an anti-FcRn antibody or an antigen-binding fragment thereof for use in a method of treating Graves' Disease. Pharmaceutical compositions comprising an anti-FcRn antibody or an antigen-binding fragment thereof and at least one pharmaceutically acceptable carrier are also disclosed and are useful in the therapeutic methods and uses described herein.

As used herein, the term “treat” and its cognates refer to an amelioration of a disease, disorder, or condition (e.g., Graves' Disease), or at least one discernible symptom thereof (e.g., any one or more of the signs and symptoms described herein). The term “treat” encompasses but is not limited to complete treatment or complete amelioration of one or more symptoms of Graves' Disease. In some embodiments, “treat” refers to at least partial amelioration of at least one measurable physical parameter, not necessarily discernible by the patient, e.g., a reduction in the level of at least one autoantibody and/or pathogenic antibody (e.g., a pathogenic IgG) and/or the level of total serum IgG. In some embodiments, “treat” refers to inhibiting the progression of Graves' Disease, either physically (e.g., stabilization of a discernible symptom), physiologically (e.g., stabilization of a physical parameter), or both. In some embodiments, “treat” refers to slowing the progression or reversing the progression of Graves' Disease.

The antibodies, antigen-binding fragments, and pharmaceutical compositions disclosed herein can also be used in the prevention or prophylaxis of Graves' Disease. For instance, a prophylactic method can comprise administering to a subject at risk of developing Graves' Disease an antibody, antigen-binding fragment, or pharmaceutical composition disclosed herein to prevent or reduce the odds developing Graves' Disease, or at least one discernible symptom thereof.

The terms “subject” and “patient” are used interchangeably herein to refer to any human or non-human animal. Non-human animals include all vertebrates (e.g., mammals and non-mammals) such as any mammal. Non-limiting examples of mammals include humans, mice, rats, rabbits, dogs, monkeys, and pigs. In various embodiments, the subject is a human. In various embodiments, the subject is a human having or suspected of having Graves' Disease.

In some embodiments, a patient in need of treatment for Graves' Disease exhibits one or more signs and symptoms of Graves' Disease (e.g., anxiety and irritability, a fine tremor of the hands or fingers, heat sensitivity or intolerance, increase in perspiration, warm, moist skin, weight loss despite normal eating habits, goiter, change in menstrual cycles, erectile dysfunction or reduced libido, frequent bowel movements, fatigue, thick red skin on the shins or tops of the feet (Graves' dermopathy), changes in hair and/or skin texture, brittle bones, heart palpitations, muscle weakness of the hips and shoulders, sleep disturbance) and/or has been diagnosed with any form of the condition by a treating clinician.

In some embodiments, a patient in need of treatment for Graves' Disease does not suffer from Graves' ophthalmology. In some embodiments, a patient in need of treatment for Graves' Disease has not received anti-FcRn antibody treatment for Graves' ophthalmology.

In some embodiments, a patient in need of treatment for Graves' Disease (or a sample from the patient) has a detectable level of an anti-thyrotropin receptor (anti-TSH-R) IgG. In some embodiments, the anti-TSH-R IgG is present in the patient's blood. In some embodiments, the anti-TSH-R IgG is an anti-TSH-R IgG1. In some embodiments, the anti-TSH-R IgG is an anti-TSH-R IgG2. In some embodiments, the anti-TSH-R IgG is an anti-TSH-R IgG3. In some embodiments, the anti-TSH-R IgG is an anti-TSH-R IgG4.

In some embodiments, a patient in need of treatment for Graves' Disease has active hyperthyroidism. In some embodiments, a patient in need of treatment for Graves' Disease has active hyperthyroidism due to Graves' Disease. Active hyperthyroidism may be determined by measuring the levels of TSH-receptor antibodies (TRAb), thyroid-simulating hormone (TSH), free triiodothyronine (FT3) and/or free thyroxine (FT4) in a sample from the patient.

In some embodiments, a patient treated in accordance with a method described herein is receiving one or antithyroid drug (ATD). In some embodiments, the patient is receiving one or more ATD but still fails to achieve euthyroidism (normal thyroid function as determined by normal serum levels of TSH, FT3 and/or FT4). In some embodiments, the normal range of TSH levels for adults is about 0.27-4.2 uIU/mL. In some embodiments, the normal range of FT3 levels for adults is about 2.3-4.1 pg/mL. In some embodiments, the normal range of FT4 levels for adults is about 0.9-1.7 ng/dL. The normal range of TSH, FT3 and/or FT4 and assays used to measure it are known in the art. In some embodiments, the normal range of TSH, FT3 and/or FT4 may differ depending on the assay used to measure it. Examples of ATDs include propylthiouracil, methimazole, and carbimazole.

In some embodiments, a patient treated in accordance with a method described herein has received an ATD for at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, or at least 20 weeks before the first administration of an anti-FcRN antibody. In some embodiments, a patient treated in accordance with a method described herein has received an ATD for at least 21 weeks, or at least 22 weeks, or at least 23 weeks, or at least 24 weeks, or at least 25 weeks, or at least 26 weeks, or at least 27 weeks, or at least 28 weeks, or at least 29 weeks, or at least 30 weeks before the first administration of an anti-FcRN antibody. In some embodiments, a patient treated in accordance with a method described herein has received an ATD for at least 6 months before the first administration of an anti-FcRN antibody.

In some embodiments, a patient treated in accordance with a method described herein has not received an ATD before the first administration of an anti-FcRN antibody. In some embodiments, a patient treated in accordance with a method described herein is contraindicated for administration of an ATD. In some embodiments, a patient treated in accordance with a method described herein has relapsed after receiving an ATD before the first administration of an anti-FcRN antibody.

In some embodiments, a patient treated in accordance with a method described herein has received starting dose of ≥20 mg, or ≥30 mg, or ≥40 mg, or ≥50 mg, or ≥60 mg of methimazole daily. In some embodiments, a patient treated in accordance with a method described herein has received starting dose of ≤20 mg, or ≤15 mg, or ≤10 mg, or ≤5 mg, or ≤2.5 mg, or ≤1 mg of methimazole daily. In some embodiments, a patient treated in accordance with a method described herein has received starting dose of ≥20 mg, or ≥30 mg, or ≥40 mg, or 50 mg, or ≥60 mg of carbimazole daily. In some embodiments, a patient treated in accordance with a method described herein has received starting dose of ≤20 mg, or ≤15 mg, or ≤10 mg, or ≤5 mg, or ≤2.5 mg, or ≤1 mg of carbimazole daily. In some embodiments, a patient treated in accordance with a method described herein has received starting dose of ≥200 mg, or ≥300 mg, or ≥400 mg, or ≥500 mg of propylthiouracil daily. In some embodiments, a patient treated in accordance with a method described herein has received starting dose of ≤200 mg, or ≤100 mg, or ≤75 mg, ≤50 mg, or ≤25 mg, or ≤10 mg of propylthiouracil daily. In some embodiments, a patient treated in accordance with a method described herein is receiving a total daily dose of ≥3 mg, or ≥5 mg, or 10 mg, or ≥20 mg, or ≥30 mg, or 40 mg, or 50 mg, or 60 mg of methimazole. In some embodiments, a patient treated in accordance with a method described herein is receiving a total daily dose of ≥3 mg, or ≥5 mg, or ≥10 mg, or ≥20 mg, or ≥30 mg, or ≥40 mg, or 50 mg, or ≥60 mg of carbimazole. In some embodiments, a patient treated in accordance with a method described herein is receiving a total daily dose of ≥30 mg, or ≥50 mg, or ≥100 mg, ≥200 mg, or ≥300 mg, or ≥400 mg, or ≥500 mg of propylthiouracil.

In some embodiments, the amount of ATD the patient is receiving is reduced after administration of the antibody or antigen-binding fragment thereof. In some embodiments, the amount of ATD is reduced by at least about 10%, at least about 20%, at least about 30%, at least about 40%, at least about 50%, at least about 60%, at least about 70%, at least about 80%, at least about 90% or at least about 95%. In some embodiments, treatment with the ATD is discontinued after the administration of the anti-FcRn antibody.

In some embodiments, a patient treated in accordance with a method described herein does not have a history of hyperthyroidism not caused by Graves' Disease (e.g., toxic adenoma or toxic multinodular goiter). In some embodiments, a patient treated in accordance with a method described herein does not have a history or presence of thyroid storm. In some embodiments, a patient treated in accordance with a method described herein has not received treatment with radioactive iodine ablation or thyroidectomy. In some embodiment, a patient treated in accordance with a method described herein has total immunoglobulin G (IgG) levels of at least 6 grams per liter (g/L) before being administered the anti-FcRn antibody. In some embodiment, a patient treated in accordance with a method described herein has albumin levels of at least 3.5 grams per deciliter (g/dL) (<35 g/L) before being administered the anti-FcRn antibody. In some embodiment, a patient treated in accordance with a method described herein has an absolute neutrophil count of at least 1000 cells per cubic millimeter (cells/mm³) before being administered the anti-FcRn antibody.

One embodiment is a method of treating Graves' Disease in a patient in need thereof, comprising administering to the patient (i) a therapeutically effective amount of an anti-FcRn antibody or an antigen-binding fragment thereof; or (ii) a pharmaceutical composition comprising at least one pharmaceutically acceptable carrier and a therapeutically effective amount of an anti-FcRn antibody or an antigen-binding fragment thereof.

Another embodiment is an anti-FcRn antibody or an antigen-binding fragment thereof for use in a method of treating Graves' Disease in a patient in need thereof, the method comprising administering to the patient (i) a therapeutically effective amount of the antibody or antigen-binding fragment, or (ii) a pharmaceutical composition comprising at least one pharmaceutically acceptable carrier and a therapeutically effective amount of the antibody or antigen-binding fragment.

Another embodiment is a use of an anti-FcRn antibody or an antigen-binding fragment thereof in a method of treating Graves' Disease in a patient in need thereof, comprising administering to the patient (i) a therapeutically effective amount of the antibody or antigen-binding fragment; or (ii) a pharmaceutical composition comprising at least one pharmaceutically acceptable carrier and a therapeutically effective amount of the antibody or antigen-binding fragment.

Another embodiment is a use of an anti-FcRn antibody or an antigen-binding fragment thereof in the manufacture of a medicament for treating Graves' Disease in a patient in need thereof.

In various embodiments of the therapeutic methods, uses, and compositions disclosed herein, an anti-FcRn antibody or antigen-binding fragment acts as a non-competitive inhibitor of IgG in binding to FcRn. In various embodiments, the binding of the antibody or antigen-binding fragment to FcRn inhibits the binding of at least one autoantibody and/or pathogenic antibody to FcRn. In various embodiments, such inhibition promotes clearance (i.e., removal) of the at least one autoantibody and/or pathogenic antibody from the body of the subject. In various embodiments, such inhibition reduces the half-life of the at least one autoantibody and/or pathogenic antibody. In various embodiments, such inhibition reduces the level of the at least one autoantibody and/or pathogenic antibody in the subject and/or in a sample from the subject. In various embodiments, a reduction in the level of the at least one autoantibody and/or pathogenic antibody results in and/or correlates with an improvement in at least one clinical parameter of Graves' Disease.

As used herein, the term “autoantibody” refers to an antibody produced by an organism's immune system that is directed against one or more of the organism's own proteins, tissues, and/or organs. For instance, one or more autoantibodies may be produced by a human patient's immune system when it fails to distinguish between “self” and “non-self.” In some embodiments, the autoantibody is a pathogenic antibody (e.g., a pathogenic IgG, e.g., a pathogenic IgG1, IgG2, IgG3, or IgG4). The term “pathogenic antibody,” as used herein, refers to an antibody (e.g., an autoantibody) that contributes to the pathogenesis of and/or causes one or more diseases, disorders, or conditions (e.g., Graves' Disease).

In some embodiments, the pathogenic antibody is a pathogenic IgG (e.g., a pathogenic IgG1, IgG2, IgG3, or IgG4). In some embodiments, the pathogenic antibody and/or pathogenic IgG is an anti-TSH-R IgG. In some embodiments, the pathogenic antibody is an anti-TSH-R IgG1.

In some embodiments, treatment of a patient with an antibody, antigen-binding fragment, or pharmaceutical composition described herein, e.g., using a method described herein, reduces the level of an anti-TSH-R IgG (e.g., an anti-TSH-R IgG1, an anti-TSH-R IgG2, an anti-TSH-R IgG3, and/or an anti-TSH-R IgG4) by least about 20%, about 25%, about 30%, about 35%, about 40%, about 45%, about 50%, i.e., relative to the level of the anti-TSH-R IgG prior to treatment.

In some embodiments, the autoantibody and/or pathogenic antibody is an IgG, IgM, IgA, IgD, or IgE. In some embodiments, the autoantibody and/or pathogenic antibody is an IgG (e.g., a pathogenic IgG). In some embodiments, the autoantibody and/or pathogenic antibody is an IgG1, IgG2, IgG3, or IgG4. In some embodiments, the autoantibody and/or pathogenic antibody is an IgG1 (e.g., a pathogenic IgG1, e.g., an anti-TSH-R IgG1). In some embodiments, the autoantibody and/or pathogenic antibody is an IgG2 (e.g., a pathogenic IgG2, e.g., an anti-TSH-R IgG2). In some embodiments, the autoantibody and/or pathogenic antibody is an IgG3 (e.g., a pathogenic IgG3, e.g., an anti-TSH-R IgG3). In some embodiments, the autoantibody and/or pathogenic antibody is an IgG4 (e.g., a pathogenic IgG4, e.g., an anti-TSH-R IgG4). In some embodiments, the autoantibody is a pathogenic antibody.

### Dosage and Administration

Dosage regimens for the anti-FcRn antibody or antigen-binding fragment, either alone or in combination with one or more additional therapeutic agents or therapy, may be adjusted to provide the optimum desired response (e.g., a therapeutic response). For example, a single bolus of the anti-FcRn antibody or antigen-binding fragment may be administered at one time, several divided doses may be administered over a predetermined period of time, or the dose of the anti-FcRn antibody or antigen-binding fragment may be proportionally reduced or increased as indicated by the exigencies of the therapeutic situation. For any particular subject, specific dosage regimens may be adjusted over time according to the individual's need, and the professional judgment of the treating clinician. For instance, in some embodiments, the dose of the anti-FcRn antibody or antigen-binding fragment can be suitably determined by taking into consideration the patient's severity, condition, age, case history, and the like.

Dosage values for the anti-FcRn antibody or antigen-binding fragment, compositions comprising the anti-FcRn antibody or antigen-binding fragment, and/or any additional therapeutic agent(s), may be selected based on the unique characteristics of the active compound(s) and the particular therapeutic effect to be achieved. A physician or veterinarian can start doses of the antibodies or antigen-binding fragments at levels lower than those required to achieve the desired therapeutic effect and gradually increase the dosage until the desired effect is achieved. A physician or veterinarian can also start doses of the antibodies or antigen-binding fragments at levels higher than those required to achieve the desired therapeutic effect and gradually decrease the dosage until the desired effect is achieved. In general, effective doses of the antibodies or antigen-binding fragments for the treatment of Graves' Disease may vary depending upon many different factors, for example, on the stage of the disease, the severity of symptoms, and the subject's prognosis. The selected dosage level may also depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. In some embodiments, the treatment may be administered once or several times. Intermittent and/or chronic (continuous) dosing strategies may be applied in view of the condition of the particular patient.

In some embodiments, a therapeutically effective amount of the anti-FcRn antibody or antigen-binding fragment is employed in the methods, uses, and pharmaceutical compositions of the present disclosure.

As used herein, the terms “therapeutically effective amount” and “therapeutically effective dose” are used interchangeably herein to refer to an amount sufficient to decrease at least one symptom or measurable parameter associated with a disease, disorder, or condition; to normalize body functions in a disease, disorder, or condition that results in the impairment of specific bodily functions; and/or to provide improvement in, or slow the progression of, one or more clinically measured parameters of a disease, disorder, or condition. A therapeutically effective amount may, for example, be sufficient to treat, reduce the severity, delay the onset, and/or reduce the risk of occurrence of one or more symptoms of Graves' Disease. A therapeutically effective amount, as well as a therapeutically effective frequency of administration, can be determined by methods known in the art and discussed herein. In some embodiments of the methods, uses, and compositions described herein, the anti-FcRn antibody or antigen-binding fragment is administered in an amount that is therapeutically effective when administered as a single agent. In some embodiments, the anti-FcRn antibody or antigen-binding fragment and at least one additional therapeutic agent are each administered in an amount that is therapeutically effective when the agents are used in combination. In some embodiments, a therapeutically effective amount of the anti-FcRn antibody or antigen-binding fragment is the amount required to reduce the level of total serum IgG and/or the level of at least one autoantibody (e.g., at least one IgG) in a patient having or suspected of having Graves' Disease.

In some embodiments, a therapeutically effective amount of the anti-FcRn antibody or antigen-binding fragment is the amount required to reduce the level of total serum IgG and/or the level of at least one autoantibody and/or pathogenic antibody (e.g., at least one IgG) in a patient with Graves' Disease and/or in a sample from the patient with Graves' Disease by at least about 20%, about 25%, about 30%, about 35%, about 40%, about 45%, about 50%, about 55%, about 60%, about 65%, about 70%, about 75%, or about 80% relative to the level prior to treatment with the anti-FcRn antibody or antigen-binding fragment. In some embodiments, a therapeutically effective amount of the anti-FcRn antibody or antigen-binding fragment is the amount required to reduce the level of total serum IgG and/or the level of at least one autoantibody and/or pathogenic antibody (e.g., at least one IgG) a patient with Graves' Disease and/or in a sample from the patient with Graves' Disease by at least about 40%, about 50%, about 60%, about 70%, or about 80% relative to the level prior to treatment with the anti-FcRn antibody or antigen-binding fragment. In some embodiments, a therapeutically effective amount of the anti-FcRn antibody or antigen-binding fragment is the amount required to reduce the serum endogenous IgG concentration in a patient with Graves' Disease and/or in a sample from the patient with Graves' Disease to less than about 40%, about 50%, about 60%, about 70%, or about 80% of pretreatment values. In some embodiments, the autoantibody is an anti-TSH-R IgG.

The phrase “total IgG level” or “level of total serum IgG,” as used herein, refers to the serum endogenous IgG concentration, e.g., in a patient or in a biological sample (e.g., a blood sample) from a patient.

The phrase “level of at least one autoantibody,” as used herein, refers to the serum endogenous concentration of the at least one autoantibody, e.g., in a patient or in a biological sample from a patient.

The phrase “level of at least one IgG,” as used herein, refers to the serum endogenous concentration of the at least one IgG, e.g., in a patient or in a biological sample from a patient. In some embodiments, the at least one IgG comprises a pathogenic IgG. In some embodiments, the at least one IgG comprises serum IgG1. In some embodiments, the at least one IgG comprises serum IgG2. In some embodiments, the at least one IgG comprises serum IgG3. In some embodiments, the at least one IgG comprises serum IgG4.

The term “about” or “approximately,” as used herein in the context of numerical values and ranges, refers to values or ranges that approximate or are close to the recited values or ranges such that the embodiment may perform as intended, as is apparent to the skilled person from the teachings contained herein. These terms encompass values beyond those resulting from systematic error. In some embodiments, “about” or “approximately” means plus or minus 10% of a numerical amount.

In some embodiments, a therapeutically effective amount of the anti-FcRn antibody or antigen-binding fragment is the amount required to reduce the level of triiodothyronine (FT3) and/or free thyroxine (FT4) in a patient with Graves' Disease and/or in a sample from the patient with Graves' Disease by at least about 20%, about 25%, about 30%, about 35%, about 40%, about 45%, about 50%, about 55%, about 60%, about 65%, about 70%, about 75%, or about 80% relative to the level prior to treatment with the anti-FcRn antibody or antigen-binding fragment. In some embodiments, a therapeutically effective amount of the anti-FcRn antibody or antigen-binding fragment is the amount required to reduce the level of FT3 and/or FT4 in a patient with Graves' Disease and/or in a sample from the patient with Graves' Disease by at least about 40%, about 50%, about 60%, about 70%, or about 80% relative to the level prior to treatment with the anti-FcRn antibody or antigen-binding fragment. In some embodiments, a therapeutically effective amount of the anti-FcRn antibody or antigen-binding fragment is the amount required to reduce the levels of FT3 and/or FT4 in a patient with Graves' Disease and/or in a sample from the patient with Graves' Disease to less than about 40%, about 50%, about 60%, about 70%, or about 80% of pretreatment values. In some embodiments, a therapeutically effective amount of the anti-FcRn antibody or antigen-binding fragment is the amount required to normalize the levels of FT3 and/or FT4 in a patient with Graves' Disease. In some embodiments, a therapeutically effective amount of the anti-FcRn antibody or antigen-binding fragment is the amount required to normalize the levels of FT3, FT4 and/or TSH in a patient with Graves' Disease. In some embodiments, a therapeutically effective amount of the anti-FcRn antibody or antigen-binding fragment is the amount required to produce seroconversion (i.e., negative for TSH receptor antibody).

In various embodiments of the therapeutic methods and uses disclosed herein, the antibody or antigen-binding fragment is administered to a patient as a fixed dose. In various embodiments of the therapeutic methods and uses disclosed herein, the antibody or antigen-binding fragment is administered to a patient as a weight-based dose, i.e., a dose dependent on the patient's bodyweight. In various embodiments of the therapeutic methods and uses disclosed herein, the antibody or antigen-binding fragment is administered to a patient as a body surface area-based dose, i.e., a dose dependent on the patient's body surface area (BSA). In various embodiments, the dose administered to the patient comprises a therapeutically effective amount of the antibody or antigen-binding fragment.

In some embodiments, the antibody or antigen-binding fragment is administered to the patient at dose of about 170 mg to about 1500 mg. In some embodiments, the antibody or antigen-binding fragment is administered to the patient at dose of about 300 mg to about 800 mg. In some embodiments, the antibody or antigen-binding fragment is administered to the patient at a dose of about 170 mg, about 200 mg, about 250 mg, about 255 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg, e.g., once weekly or once every 2 weeks.

In some embodiments, the antibody or antigen-binding fragment is administered to the patient at a dose of about 100 mg to about 200 mg. In some embodiments, the antibody or antigen-binding fragment is administered to the patient at a dose of about 100 mg, about 110 mg, about 120 mg, about 130 mg, about 140 mg, about 150 mg, about 160 mg, about 170 mg, about 180 mg, about 190 mg, or about 200 mg.

In some embodiments, the antibody or antigen-binding fragment is administered to the patient at a dose of about 170 mg to about 300 mg. In some embodiments, the antibody or antigen-binding fragment is administered to the patient at a dose of about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg.

In some embodiments, the antibody or antigen-binding fragment is administered to the patient at a dose of about 300 mg to about 500 mg. In some embodiments, the antibody or antigen-binding fragment is administered to the patient at a dose of about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg.

In some embodiments, the antibody or antigen-binding fragment is administered to the patient at a dose of about 300 mg to about 400 mg. In some embodiments, the antibody or antigen-binding fragment is administered to the patient at a dose of about 300 mg, about 310 mg, about 320 mg, about 330 mg, about 340 mg, about 350 mg, about 360 mg, about 370 mg, about 380 mg, about 390 mg, or about 400 mg. In some embodiments, the antibody or antigen-binding fragment is administered to the patient at a dose of about 320 mg, about 330 mg, about 340 mg, about 350 mg, or about 360 mg. In some embodiments, the antibody or antigen-binding fragment is administered to the patient at a dose of about 340 mg. In some embodiments, the antibody or antigen-binding fragment is administered to the patient at a dose of about 340 mg once weekly or once every 2 weeks. In some embodiments, the antibody or antigen-binding fragment is administered to the patient at a dose of about 340 mg once weekly. In some embodiments, the antibody or antigen-binding fragment is administered to the patient at a dose of about 340 mg once weekly as a single subcutaneous injection. In some embodiments, the antibody or antigen-binding fragment is administered to the patient at a dose of about 340 mg once weekly for at least 2 weeks (e.g., 2 weeks, 3 weeks, 4 weeks, 5 weeks, 6 weeks, 7 weeks, 8 weeks, 10 weeks, 12 weeks, or longer). In some embodiments, the antibody or antigen-binding fragment is administered to the patient at a dose of about 340 mg once weekly for at least 4 weeks. In some embodiments, the antibody or antigen-binding fragment is administered to the patient at a dose of about 340 mg once weekly for at least 7 weeks. In some embodiments, the antibody or antigen-binding fragment is administered to the patient at a dose of about 340 mg once weekly for at least 12 weeks.

In some embodiments, the antibody or antigen-binding fragment is administered to the patient at a dose of about 500 mg to about 700 mg. In some embodiments, the antibody or antigen-binding fragment is administered to the patient at a dose of about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg.

In some embodiments, the antibody or antigen-binding fragment is administered to the patient at a dose of about 650 mg to about 750 mg. In some embodiments, the antibody or antigen-binding fragment is administered to the patient at a dose of about 650 mg, about 660 mg, about 670 mg, about 680 mg, about 690 mg, about 700 mg, about 710 mg, about 720 mg, about 730 mg, about 740 mg, or about 750 mg. In some embodiments, the antibody or antigen-binding fragment is administered to the patient at a dose of about 660 mg, about 670 mg, about 680 mg, about 690 mg, or about 700 mg. In some embodiments, the antibody or antigen-binding fragment is administered to the patient at a dose of about 680 mg. In some embodiments, the antibody or antigen-binding fragment is administered to the patient at a dose of about 680 mg once weekly or once every 2 weeks. In some embodiments, the antibody or antigen-binding fragment is administered to the patient at a dose of about 680 mg once weekly. In some embodiments, the antibody or antigen-binding fragment is administered to the patient at a dose of about 680 mg once weekly as two or more (e.g., two) consecutive subcutaneous injections. In some embodiments, the antibody or antigen-binding fragment is administered to the patient at a dose of about 680 mg once weekly for at least 2 weeks (e.g., 2 weeks, 3 weeks, 4 weeks, 5 weeks, 6 weeks, 7 weeks, 8 weeks, 10 weeks, 12 weeks, or longer). In some embodiments, the antibody or antigen-binding fragment is administered to the patient at a dose of about 680 mg once weekly for at least 4 weeks. In some embodiments, the antibody or antigen-binding fragment is administered to the patient at a dose of about 680 mg once weekly for at least 7 weeks. In some embodiments, the antibody or antigen-binding fragment is administered to the patient at a dose of about 680 mg once weekly for at least 12 weeks.

In some embodiments, the antibody or antigen-binding fragment is administered to the patient at a dose of about 700 mg to about 900 mg. In some embodiments, the antibody or antigen-binding fragment is administered to the patient at a dose of about 700 mg, about 710 mg, about 720 mg, about 730 mg, about 740 mg, about 750 mg, about 760 mg, about 765 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, or about 900 mg.

In some embodiments, the antibody or antigen-binding fragment is administered to the patient at a dose of about 900 mg to about 1100 mg. In some embodiments, the antibody or antigen-binding fragment is administered to the patient at a dose of about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, or about 1100 mg.

In some embodiments, the antibody or antigen-binding fragment is administered to the patient at a dose of about 1100 mg to about 1300 mg. In some embodiments, the antibody or antigen-binding fragment is administered to the patient at a dose of about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, or about 1300 mg.

In some embodiments, the antibody or antigen-binding fragment is administered to the patient at a dose of about 1300 mg to about 1500 mg. In some embodiments, the antibody or antigen-binding fragment is administered to the patient at a dose of about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, or about 1500 mg.

In some embodiments, the antibody or antigen-binding fragment is administered to the patient at a dose of about 1 mg/kg to about 2000 mg/kg bodyweight. In some embodiments, the antibody or antigen-binding fragment is administered to the patient at a dose of about 1 mg/kg to about 200 mg/kg, about 200 mg/kg to about 400 mg/kg, about 400 mg/kg to about 600 mg/kg, about 600 mg/kg to about 800 mg/kg, about 800 mg/kg to about 1000 mg/kg, about 1000 mg/kg to about 1200 mg/kg, about 1200 mg/kg to about 1400 mg/kg, about 1400 mg/kg to about 1600 mg/kg, about 1600 mg/kg to about 1800 mg/kg, or about 1800 mg/kg to about 2000 mg/kg. In some embodiments, the antibody or antigen-binding fragment is administered to the patient at a dose of about 1 mg/kg to about 200 mg/kg. In some embodiments, the antibody or antigen-binding fragment is administered to the patient at a dose of about 1 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the antibody or antigen-binding fragment is administered to the patient at a dose of about 1 mg/kg to about 40 mg/kg. In some embodiments, the antibody or antigen-binding fragment is administered to the patient at a dose of about 1 mg/kg, about 5 mg/kg, about 10 mg/kg, about 15 mg/kg, about 20 mg/kg, about 25 mg/kg, about 30 mg/kg, about 35 mg/kg, or about 40 mg/kg.

The frequency with which the antibody or antigen-binding fragment is administered to the patient, as a single agent or in combination with one or more additional therapeutic agents, may be once or more than once. In some embodiments, the antibody or antigen-binding fragment is administered on a single occasion. In some embodiments, the antibody or antigen-binding fragment is administered on multiple occasions. Intervals between dosages can be, e.g., daily, weekly, bi-weekly, monthly, or yearly. Intervals can also be irregular, e.g., based on measuring blood levels of the antibody or antigen-binding fragment in the patient in order to maintain a relatively consistent plasma concentration of the antibody or antigen-binding fragment; based on measuring levels of at least one autoantibody and/or pathogenic antibody (e.g., at least one IgG) in order to maintain a reduced level of the at least one autoantibody and/or pathogenic antibody (e.g., the at least one IgG) so as to provide the desired therapeutic or prophylactic effect; based on measuring levels of total serum IgG in order to maintain a reduced level of total serum IgG so as to provide the desired therapeutic or prophylactic effect; and/or based on measuring levels of hemoglobin in order to maintain an increased level of hemoglobin so as to provide the desired therapeutic or prophylactic effect. Alternatively, in some embodiments, the antibody or antigen-binding fragment can be administered as a sustained release formulation, in which case less frequent administration is required. Dosage and frequency may vary depending on the half-life of the antibody or antigen-binding fragment in the patient. The dosage and frequency of administration may also vary depending on whether the treatment is prophylactic or therapeutic. In prophylactic applications, a relatively low dosage may be administered at relatively infrequent intervals over a long period of time. Some patients may continue to receive treatment for the rest of their lives. In therapeutic applications, a relatively higher dosage at relatively shorter intervals is sometimes administered until progression of the disease is reduced or terminated, and optionally until the patient shows partial or complete amelioration of one or more symptoms of the disease. Thereafter, the patient may be administered a lower, e.g., prophylactic regime.

In some embodiments, the antibody, antigen-binding fragment, or pharmaceutical composition is administered to the patient once or more than once over a period of about 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 1 month, 2 months, 3 months, 4 months, 5 months, 6 months, 7 months, 8 months, 9 months, 10 months, 11 months, 12 months, 18 months, 24 months, 30 months, 36 months, 48 months, 52 months, 60 months, or longer.

In some embodiments, the antibody, antigen-binding fragment, or pharmaceutical composition is administered to the patient once as a single dose.

In some embodiments, the antibody, antigen-binding fragment, or pharmaceutical composition is administered to the patient once weekly. In some embodiments, the antibody, antigen-binding fragment, or pharmaceutical composition is administered to the patient once weekly for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 12 weeks, at least 20 weeks, at least 24 weeks, at least 30 weeks, at least 40 weeks, at least 50 weeks, at least 60 weeks, at least 70 weeks, at least 76 weeks, at least 80 weeks, or longer. In some embodiments, the antibody, antigen-binding fragment, or pharmaceutical composition is administered to the patient once weekly for 6 to 76 weeks, or any time period in between. In some embodiments, the antibody, antigen-binding fragment, or pharmaceutical composition is administered to the patient once weekly for at least 2 weeks, at least 3 weeks, at least 4 weeks, or at least 6 weeks. In some embodiments, the antibody, antigen-binding fragment, or pharmaceutical composition is administered to the patient once weekly for at least 4 weeks. In some embodiments, the antibody, antigen-binding fragment, or pharmaceutical composition is administered to the patient once weekly for at least 7 weeks. In some embodiments, the antibody, antigen-binding fragment, or pharmaceutical composition is administered to the patient once weekly for at least 12 weeks. In some embodiments, the antibody, antigen-binding fragment, or pharmaceutical composition is administered to the patient once weekly for at least 24 weeks. In some embodiments, the antibody, antigen-binding fragment, or pharmaceutical composition is administered to the patient once weekly for at least 52 weeks. In some embodiments, the antibody, antigen-binding fragment, or pharmaceutical composition is administered to the patient once weekly until sufficient to treat, prevent, reduce the severity, delay the onset, and/or reduce the risk of occurrence of one or more symptoms of Graves' Disease.

In some embodiments, the antibody, antigen-binding fragment, or pharmaceutical composition is administered to the patient once weekly as a single (i.e., one) subcutaneous injection. In some embodiments, the antibody, antigen-binding fragment, or pharmaceutical composition is administered to the patient once weekly as two or more consecutive subcutaneous injections (e.g., two consecutive subcutaneous injections). The term “consecutive,” as used herein in the context of subcutaneous injections (or other routes of administration), refers to two or more subcutaneous injections administered one after another, but sufficiently close in time so as to provide the desired therapeutic or prophylactic effect. In some embodiments, consecutive subcutaneous injections are administered within about 30 seconds, within about 1 minute, within about 2 minutes, within about 5 minutes, within about 10 minutes, within about 30 minutes, within about 1 hour, within about 2 hours, or within about 5 hours of one another.

In some embodiments, the antibody, antigen-binding fragment, or pharmaceutical composition is administered to the patient once every 2 weeks (bi-weekly). In some embodiments, the antibody, antigen-binding fragment, or pharmaceutical composition is administered to the patient once every 2 weeks for at least 2 weeks, at least 4 weeks, at least 6 weeks, at least 8 weeks, at least 10 weeks, at least 12 weeks, at least 20 weeks, at least 24 weeks, at least 30 weeks, at least 40 weeks, at least 50 weeks, at least 60 weeks, at least 70 weeks, at least 76 weeks, at least 80 weeks, or longer. In some embodiments, the antibody, antigen-binding fragment, or pharmaceutical composition is administered to the patient once every 2 weeks for 6 to 76 weeks, or any time period in between. In some embodiments, the antibody, antigen-binding fragment, or pharmaceutical composition is administered to the patient once every 2 weeks for at least 12 weeks. In some embodiments, the antibody, antigen-binding fragment, or pharmaceutical composition is administered to the patient weekly for at least 12 weeks. In some embodiments, the antibody, antigen-binding fragment, or pharmaceutical composition is administered to the patient weekly for at least 24 weeks. In some embodiments, the antibody, antigen-binding fragment, or pharmaceutical composition is administered to the patient weekly for at least 52 weeks. In some embodiments, the antibody, antigen-binding fragment, or pharmaceutical composition is administered to the patient weekly for up to 24 weeks. In some embodiments, the antibody, antigen-binding fragment, or pharmaceutical composition is administered to the patient weekly for up to 52 weeks. In some embodiments, the antibody, antigen-binding fragment, or pharmaceutical composition is administered to the patient weekly until sufficient to treat, prevent, reduce the severity, delay the onset, and/or reduce the risk of occurrence of one or more symptoms of Graves' Disease. In some embodiments, the antibody, antigen-binding fragment, or pharmaceutical composition is administered to the patient weekly as a single subcutaneous injection. In some embodiments, the antibody, antigen-binding fragment, or pharmaceutical composition is administered to the patient once weekly as two or more consecutive subcutaneous injections.

In some embodiments, the antibody, antigen-binding fragment, or pharmaceutical composition is administered to the patient once monthly. In some embodiments, the antibody, antigen-binding fragment, or pharmaceutical composition is administered to the patient once monthly for at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or longer. In some embodiments, the antibody, antigen-binding fragment, or pharmaceutical composition is administered to the patient once monthly until sufficient to treat, prevent, reduce the severity, delay the onset, and/or reduce the risk of occurrence of one or more symptoms of Graves' Disease. In some embodiments, the antibody, antigen-binding fragment, or pharmaceutical composition is administered to the patient once monthly as a single subcutaneous injection. In some embodiments, the antibody, antigen-binding fragment, or pharmaceutical composition is administered to the patient once monthly as two or more consecutive subcutaneous injections.

In some embodiments of the therapeutic methods, uses, and compositions disclosed herein, the therapeutically effective amount of the antibody or antigen-binding fragment is about 170 mg to about 1500 mg administered once as a single dose. More specifically, in some embodiments, the therapeutically effective amount of the antibody or antigen-binding fragment is about 170 mg to about 300 mg administered once as a single dose. In some embodiments, the therapeutically effective amount of the antibody or antigen-binding fragment is about 300 mg to about 500 mg administered once as a single dose. In some embodiments, the therapeutically effective amount of the antibody or antigen-binding fragment is about 500 mg to about 700 mg administered once as a single dose. In some embodiments, the therapeutically effective amount of the antibody or antigen-binding fragment is about 700 mg to about 900 mg administered once as a single dose. In some embodiments, the therapeutically effective amount of the antibody or antigen-binding fragment is about 900 mg to about 1100 mg administered once as a single dose. In some embodiments, the therapeutically effective amount of the antibody or antigen-binding fragment is about 1100 mg to about 1300 mg administered once as a single dose. In some embodiments, the therapeutically effective amount of the antibody or antigen-binding fragment is about 1300 mg to about 1500 mg administered once as a single dose.

In some embodiments of the therapeutic methods, uses, and compositions disclosed herein, the therapeutically effective amount of the antibody or antigen-binding fragment is about 300 mg to about 800 mg administered once as a single dose. In some embodiments, the therapeutically effective amount of the antibody or antigen-binding fragment is about 300 mg to about 400 mg administered once as a single dose. In some embodiments, the therapeutically effective amount of the antibody or antigen-binding fragment is about 340 mg administered once as a single dose. In some embodiments, the therapeutically effective amount of the antibody or antigen-binding fragment is about 450 mg to about 550 mg administered once as a single dose. In some embodiments, the therapeutically effective amount of the antibody or antigen-binding fragment is about 500 mg administered once as a single dose. In some embodiments, the therapeutically effective amount of the antibody or antigen-binding fragment is about 700 mg to about 800 mg administered once as a single dose. In some embodiments, the therapeutically effective amount of the antibody or antigen-binding fragment is about 765 mg administered once as a single dose. In some embodiments, treatment reduces the level of total serum IgG in a patient by at least about 20%, about 25%, about 30%, about 35%, about 40%, about 45%, or about 50%. In some embodiments, treatment reduces the level of total serum IgG in a patient by at least about 25%. In some embodiments, treatment reduces the level of total serum IgG in a patient by at least about 35%. In some embodiments, treatment reduces the level of total serum IgG in a patient by at least about 45%. In some embodiments, maximum reduction in the level of total serum IgG in a patient occurs at about 5 days to about 20 days after administration of the antibody or antigen-binding fragment, or a pharmaceutical composition comprising the antibody or antigen-binding fragment. In some embodiments, maximum reduction in the level of total serum IgG in a patient occurs at about 8 days to about 15 days after administration of the antibody or antigen-binding fragment, or a pharmaceutical composition comprising the antibody or antigen-binding fragment. n some embodiments, maximum reduction in the level of total serum IgG occurs after about 3 to 5 doses (e.g., after about 4 doses) of the antibody or antigen-binding fragment, or a pharmaceutical composition comprising the antibody or antigen-binding fragment. In some embodiments, treatment increases the level of hemoglobin in a patient by at least about 5%, about 10%, about 15%, or about 20% (e.g., about 5% to about 30%). In some embodiments, treatment increases the level of hemoglobin in a patient by at least about 10% (e.g., about 10% to about 15%). In some embodiments, treatment increases the level of hemoglobin in a patient by at least about 20% (e.g., about 20% to about 25%). In some embodiments, treatment increases the level of hemoglobin in a patient by more than about 20% (e.g., about 25%, about 30%, or more).

In some embodiments of the therapeutic methods, uses, and compositions disclosed herein, the therapeutically effective amount of the antibody or antigen-binding fragment is about 170 mg to about 1500 mg administered once weekly. In some embodiments, the therapeutically effective amount of the antibody or antigen-binding fragment is about 170 mg to about 300 mg administered once weekly. In some embodiments, the therapeutically effective amount of the antibody or antigen-binding fragment is about 300 mg to about 500 mg administered once weekly. In some embodiments, the therapeutically effective amount of the antibody or antigen-binding fragment is about 500 mg to about 700 mg administered once weekly. In some embodiments, the therapeutically effective amount of the antibody or antigen-binding fragment is about 700 mg to about 900 mg administered once weekly. In some embodiments, the therapeutically effective amount of the antibody or antigen-binding fragment is about 900 mg to about 1100 mg administered once weekly. In some embodiments, the therapeutically effective amount of the antibody or antigen-binding fragment is about 1100 mg to about 1300 mg administered once weekly. In some embodiments, the therapeutically effective amount of the antibody or antigen-binding fragment is about 1300 mg to about 1500 mg administered once weekly.

In some embodiments of the therapeutic methods, uses, and compositions disclosed herein, the therapeutically effective amount of the antibody or antigen-binding fragment is about 300 mg to about 800 mg administered once weekly. In some embodiments, the therapeutically effective amount of the antibody or antigen-binding fragment is about 300 mg to about 400 mg administered once weekly. In some embodiments, the therapeutically effective amount of the antibody or antigen-binding fragment is about 340 mg administered once weekly. In some embodiments, the therapeutically effective amount of the antibody or antigen-binding fragment is about 650 mg to about 750 mg administered once weekly. In some embodiments, the therapeutically effective amount of the antibody or antigen-binding fragment is about 680 mg administered once weekly. In some embodiments, treatment reduces the level of total serum IgG in a patient by at least about 50%, about 55%, about 60%, about 65%, about 70%, about 75%, or about 80%. In some embodiments, treatment reduces the level of total serum IgG in a patient by at least about 60%. In some embodiments, treatment reduces the level of total serum IgG in a patient by at least about 70%. In some embodiments, treatment reduces the level of total serum IgG in a patient by at least about 80%. In some embodiments, maximum reduction in the level of total serum IgG in a patient occurs at about 20 days to about 30 days after administration of the antibody or antigen-binding fragment, or a pharmaceutical composition comprising the antibody or antigen-binding fragment. In some embodiments, maximum reduction in the level of total serum IgG in a patient occurs at about 24 days after administration of the antibody or antigen-binding fragment, or a pharmaceutical composition comprising the antibody or antigen-binding fragment. In some embodiments, maximum reduction in the level of total serum IgG occurs after about 3 to 5 doses (e.g., after about 4 doses) of the antibody or antigen-binding fragment, or a pharmaceutical composition comprising the antibody or antigen-binding fragment. In some embodiments, treatment increases the level of hemoglobin in a patient by at least about 5%, about 10%, about 15%, or about 20% (e.g., about 5% to about 30%). In some embodiments, treatment increases the level of hemoglobin in a patient by more than about 20%. In some embodiments, treatment increases the level of hemoglobin in a patient by at least about 10% (e.g., about 10% to about 15%) after about 1 or 2 weeks of weekly dosing (e.g., 680 mg administered once weekly). In some embodiments, treatment increases the level of hemoglobin in a patient by at least about 20% (e.g., about 20% to about 25%) after about 1 or 2 weeks of weekly dosing (e.g., 680 mg administered once weekly or 340 mg administered once weekly). In some embodiments, an increase in the level of hemoglobin in a patient (e.g., an increase of about 10%, about 20%, or more) is maintained during the entire treatment period or a portion thereof. In some embodiments, an increase in the level of hemoglobin in a patient (e.g., an increase of about 10%, about 20%, or more) is maintained for at least 4 weeks (e.g., at least 4 weeks, 6 weeks, 8 weeks, 10 weeks, 12 weeks, or longer).

In some embodiments of the therapeutic methods, uses, and compositions disclosed herein, the therapeutically effective amount of the antibody or antigen-binding fragment is about 170 mg to about 1500 mg administered once every 2 weeks. In some embodiments, the therapeutically effective amount of the antibody or antigen-binding fragment is about 300 mg to about 800 mg administered once every 2 weeks. In some embodiments, the therapeutically effective amount of the antibody or antigen-binding fragment is about 170 mg to about 300 mg administered once every 2 weeks. In some embodiments, the therapeutically effective amount of the antibody or antigen-binding fragment is about 300 mg to about 500 mg administered once every 2 weeks. In some embodiments, the therapeutically effective amount of the antibody or antigen-binding fragment is about 500 mg to about 700 mg administered once every 2 weeks. In some embodiments, the therapeutically effective amount of the antibody or antigen-binding fragment is about 700 mg to about 900 mg administered once every 2 weeks. In some embodiments, the therapeutically effective amount of the antibody or antigen-binding fragment is about 900 mg to about 1100 mg administered once every 2 weeks. In some embodiments, the therapeutically effective amount of the antibody or antigen-binding fragment is about 1100 mg to about 1300 mg administered once every 2 weeks. In some embodiments, the therapeutically effective amount of the antibody or antigen-binding fragment is about 1300 mg to about 1500 mg administered once every 2 weeks.

In some embodiments of the therapeutic methods, uses, and compositions disclosed herein, the therapeutically effective amount of the antibody or antigen-binding fragment is about 170 mg to about 1500 mg administered once monthly. In some embodiments, the therapeutically effective amount of the antibody or antigen-binding fragment is about 300 mg to about 800 mg administered once monthly. In some embodiments, the therapeutically effective amount of the antibody or antigen-binding fragment is about 170 mg to about 300 mg administered once monthly. In some embodiments, the therapeutically effective amount of the antibody or antigen-binding fragment is about 300 mg to about 500 mg administered once monthly. In some embodiments, the therapeutically effective amount of the antibody or antigen-binding fragment is about 500 mg to about 700 mg administered once monthly. In some embodiments, the therapeutically effective amount of the antibody or antigen-binding fragment is about 700 mg to about 900 mg administered once monthly. In some embodiments, the therapeutically effective amount of the antibody or antigen-binding fragment is about 900 mg to about 1100 mg administered once monthly. In some embodiments, the therapeutically effective amount of the antibody or antigen-binding fragment is about 1100 mg to about 1300 mg administered once monthly. In some embodiments, the therapeutically effective amount of the antibody or antigen-binding fragment is about 1300 mg to about 1500 mg administered once monthly.

In some embodiments of the therapeutic methods, uses, and compositions disclosed herein, the therapeutically effective amount of an antibody or antigen-binding fragment is about 340 mg or about 680 mg administered once weekly. In some embodiments, the antibody or antigen-binding fragment is administered subcutaneously.

In some embodiments, treatment with the antibody or antigen-binding fragment at a dose of about 340 mg or about 680 mg administered once weekly reduces the level of total serum IgG in a patient and/or a sample from a patient by at least about 40% (e.g., about 40% to about 50%) after about 1 or 2 weeks of weekly dosing, relative to the level of total serum IgG in the patient and/or sample prior to treatment. In some embodiments, treatment with the antibody or antigen-binding fragment at a dose of about 340 mg or about 680 mg administered once weekly reduces the level of total serum IgG in a patient and/or a sample from a patient by at least about 60% (e.g., about 60% to about 70%) after about 3 weeks of weekly dosing, relative to the level of total serum IgG in the patient and/or sample prior to treatment. In some embodiments, treatment with the antibody or antigen-binding fragment at a dose of about 340 mg or about 680 mg administered once weekly reduces the level of total serum IgG in a patient and/or a sample from a patient by at least about 70% (e.g., about 70% to about 80%) after about 5 weeks of weekly dosing, relative to the level of total serum IgG in the patient and/or sample prior to treatment. In some embodiments, the therapeutically effective amount of the antibody or antigen-binding fragment is about 680 mg administered once weekly. In some embodiments, the therapeutically effective amount of the antibody or antigen-binding fragment is about 680 mg administered once weekly for at least 2 weeks (e.g., 2 weeks, 3 weeks, 4 weeks, 5 weeks, 6 weeks, 7 weeks, 8 weeks, 10 weeks, 12 weeks, 24 weeks, 48 weeks, 52 weeks, or longer). In some embodiments, the therapeutically effective amount of the antibody or antigen-binding fragment is about 340 mg administered once weekly. In some embodiments, the therapeutically effective amount of the antibody or antigen-binding fragment is about 680 mg administered once weekly for at least 2 weeks (e.g., 2 weeks, 3 weeks, 4 weeks, 5 weeks, 6 weeks, 7 weeks, 8 weeks, 10 weeks, 12 weeks, 24 weeks, 48 weeks, 52 weeks, or longer).

In some embodiments, the antibody or antigen-binding fragment is administered to the patient at one or more doses (e.g., two or more different doses). For example, in some embodiments, the antibody or antigen-binding fragment is administered to the patient at two different doses, e.g., at least one higher dose, followed by at least one lower dose. A higher dose (e.g., a higher dose of two different doses) may be referred to herein as an “induction” dose, i.e., a dose capable of reducing the level of at least one autoantibody and/or pathogenic antibody (e.g., at least one IgG) in a patient and/or a sample from a patient. A lower dose (e.g., a lower dose of two different doses) may be referred to herein as a “maintenance” dose, i.e., a dose capable of maintaining the reduced level of at least one autoantibody and/or pathogenic antibody (e.g., at least one IgG) in the patient and/or a sample from the patient following at least one induction dose of an antibody or antigen-binding fragment (e.g., about 20-80% of pretreatment (pre-induction dose) values). In some embodiments, a maintenance dose maintains the level of at least one autoantibody and/or pathogenic antibody (e.g., at least one IgG) in the patient and/or a sample from the patient at about 20%, about 25%, about 30%, about 35%, about 40%, about 45%, about 50%, about 55%, about 60%, about 65%, about 70%, about 75%, or about 80% of pretreatment (pre-induction dose) values.

In some embodiments, the induction dose and the maintenance dose are alternated, e.g., the patient may receive the induction dose for a first period of time followed by the maintenance dose for a first period of time, followed by the induction dose for a second period of time, optionally followed by the maintenance dose for a second period of time. In some embodiments, the cycle of induction and maintenance dose (administration of the induction dose for a period of time followed by administration of the maintenance dose for a period of time being one cycle) is repeated one twice, three times, four times, five times, six times seven times, or ten times. In some embodiments, the cycle of induction and maintenance dose is repeated for about 3 months, about 6 months, about 12 months, about 15 moths, about 18 months, about 24 months, or longer.

In some embodiments, at least one higher dose and/or induction dose is about 600 mg per dose or about 680 mg per dose or more (e.g., about 700 mg per dose, about 720 mg per dose, about 750 mg per dose, or more). In some embodiments, the at least one higher dose and/or induction dose is about one dose, about 2 doses, about 3 doses, about 4 doses, or about 5 doses at about 600 mg per dose or about 680 mg per dose or more (e.g., about 700 mg per dose, about 720 mg per dose, about 750 mg per dose, or more). In some embodiments, the at least one higher dose and/or induction dose is about 3 doses at about 600 mg per dose or about 680 mg per dose or more (e.g., about 700 mg per dose, about 720 mg per dose, about 750 mg per dose, or more).

In some embodiments, the at least one higher dose and/or induction dose is administered to the patient once, once weekly, once every 2 weeks, or once monthly. In some embodiments, the at least one higher dose and/or induction dose is administered to the patient intravenously. In some embodiments, the at least one higher dose and/or induction dose is administered to the patient subcutaneously. In some embodiments, each higher dose is administered to the patient as one or more subcutaneous injections. In some embodiments, each higher dose is administered to the patient as two consecutive subcutaneous injections.

In some embodiments, the higher dose is 680 mg and is administered subcutaneously once a week for 12 weeks. In some embodiments, the higher dose is 600 mg and is administered subcutaneously once a week for 12 weeks.

In some embodiments, at least one lower dose and/or maintenance dose is about 340 mg per dose. In some embodiments, at least one lower dose and/or maintenance dose is about 300 mg per dose. In some embodiments, the at least one lower dose and/or maintenance dose is about one dose, about 2 doses, about 3 doses, about 4 doses, or about 5 doses at about 300 mg per dose or about 340 mg per dose. In some embodiments, the at least one lower dose and/or maintenance dose is about 3 doses at about 300 mg per dose or about 340 mg per dose. In some embodiments, the at least one lower dose and/or maintenance dose is administered to the patient once, once weekly, once every 2 weeks, or once monthly. In some embodiments, the at least one lower dose and/or maintenance dose is administered to the patient subcutaneously. In some embodiments, each lower dose is administered to the patient as one or more subcutaneous injections. In some embodiments, each lower dose is administered to the patient as one subcutaneous injection.

In some embodiments, the lower dose is 340 mg and is administered subcutaneously once a week for 12 weeks. In some embodiments, the lower dose is 300 mg and is administered subcutaneously once a week for 12 weeks.

In another aspect, a method described here comprises (i) administering to a subject 680 mg of an anti-FcRn antibody subcutaneously once weekly for 12 weeks, followed by (ii) administering to the subject 340 mg of the anti-FcRn antibody subcutaneously once weekly for 12 weeks. In another aspect, a method described here comprises (i) administering to a subject 600 mg of an anti-FcRn antibody subcutaneously once weekly for 12 weeks, followed by (ii) administering to the subject 300 mg of the anti-FcRn antibody subcutaneously once weekly for 12 weeks. These steps (i) and (ii) may be repeated as needed, for example, repeated twice (for a total treatment time of 48 weeks) three times(for a total treatment time of 72 weeks), four times (for a total treatment time of 96 weeks), or five times (for a total treatment time of 120 weeks).

### Combination Therapy

In various embodiments of the therapeutic methods and uses disclosed herein, the antibody or antigen-binding fragment is administered alone. In various embodiments, the antibody or antigen-binding fragment is administered in combination with at least one additional therapeutic agent. In various embodiments, the at least one additional therapeutic agent may comprise or consist of a standard-of-care agent or treatment for Graves' Disease.

Administered “in combination” or “co-administration,” as used herein, means that two or more different treatments are delivered to a subject during the subject's affliction with Graves' Disease. For example, in some embodiments, the two or more treatments are delivered after the subject has been diagnosed with the disease, and before the disease has been cured or eliminated, or when a subject is identified as being at risk but before the subject has developed symptoms of the disease. In some embodiments, the delivery of one treatment is still occurring when the delivery of the second treatment begins, so that there is overlap. In some embodiments, the first and second treatment are initiated at the same time. These types of delivery are sometimes referred to herein as “simultaneous,” “concurrent,” or “concomitant” delivery. In other embodiments, the delivery of one treatment ends before delivery of the second treatment begins. This type of delivery is sometimes referred to herein as “successive” or “sequential” delivery. In some embodiments, the antibody or antigen-binding fragment and the at least at one additional therapeutic agent are administered simultaneously. In some embodiments, the antibody or antigen-binding fragment and the at least at one additional therapeutic agent are administered sequentially.

In some embodiments, the two treatments (e.g., an anti-FcRn antibody or antigen-binding fragment and a second therapeutic agent) are present in the same composition. Such compositions may be administered in any appropriate form and by any suitable route. In other embodiments, the two treatments (e.g., an anti-FcRn antibody or antigen-binding fragment and a second therapeutic agent) are administered in separate compositions, in any appropriate form and by any suitable route. For example, a composition comprising an anti-FcRn antibody or antigen-binding fragment and a composition comprising a second therapeutic agent (e.g., a standard-of-care treatment for Graves' Disease) may be administered concurrently or sequentially, in any order at different points in time; in either case, they should be administered sufficiently close in time so as to provide the desired therapeutic or prophylactic effect.

The term “agent,” as used herein, refers to a chemical compound, a mixture of chemical compounds, a biological macromolecule, or an extract made from biological materials. The term “therapeutic agent” or “drug” refers to an agent that is capable of modulating a biological process and/or has biological activity. The anti-FcRn antibodies and antigen-binding fragments described herein are examples of therapeutic agents.

As used herein, the term “standard-of-care agent” refers to any therapeutic agent or other form of therapy that is accepted as a proper treatment for a certain type of disease (e.g., warm autoimmune hemolytic anemia). The term “standard dosage” or “standard dosing regimen,” as used herein, refers to any usual or routine dosing regimen for a therapeutic agent, e.g., a regimen proposed by the manufacturer, approved by regulatory authorities, or otherwise tested in human subjects to meet the average patient's needs.

Examples of standard of care agents for Graves' Disease include radioactive iodine, propylthiouracil, methimazole, beta blockers, and a thyroidectomy.

The anti-FcRn antibodies and antigen-binding fragments described herein may be administered in combination with any of the exemplary standard-of-care agents or therapies described herein.

### Anti-FcRn Antibodies

In various embodiments of the therapeutic methods, uses, and compositions disclosed herein, an anti-FcRn antibody or antigen-binding fragment can non-competitively inhibit the binding of at least one autoantibody and/or pathogenic antibody (e.g., at least one IgG) to FcRn at physiological pH (i.e., pH 7.0-7.4). Without wishing to be bound by theory, it is believed that FcRn binds to its ligand (i.e., IgG) and does not substantially show affinity for IgG at physiological pH rather than acidic pH. Thus, in various embodiments, at physiological pH, the anti-FcRn antibody or antigen-binding fragment may act as a non-competitive inhibitor of the binding of IgG to FcRn, and the binding of the anti-FcRn antibody or antigen-binding fragment to FcRn is not influenced by the presence of IgG. Thus, in various embodiments, the anti-FcRn antibody or antigen-binding fragment that binds specifically to FcRn non-competitively with IgG in a pH-independent manner has an advantage over conventional competitive inhibitors (i.e., antibodies that bind to FcRn competitively with IgG) in that it can provide therapeutic or prophylactic effects even at significantly low concentrations by the FcRn-mediated signaling of IgG. In addition, in various embodiments, in the procedure of intracellular migration in a state bound to FcRn, the anti-FcRn antibody or antigen-binding fragment can maintain its binding to FcRn with an affinity higher than IgG in blood. Thus, in various embodiments, the anti-FcRn antibody or antigen-binding fragment can inhibit the binding of IgG to FcRn even in endosomes that are acidic pH environments in which IgG can bind to FcRn, thereby promoting the clearance of IgG. In various embodiments, the anti-FcRn antibody or antigen-binding fragment is RVT-1401 (also referred to herein as HL161BKN and batoclimab). In some embodiments, the antibody or antigen-binding fragment is RVT-1401, or an antigen-binding fragment thereof. RVT-1401/batoclimab is also described in, e.g., international patent application publication WO2015/167293 (where it is referred to as HL161BKN), which is incorporated by reference herein in its entirety.

Illustrative CDR sequences of anti-FcRn antibodies are set forth in Table 1

TABLE 1
CDR sequences of heavy chain and light chain variable domains of selected human
FcRn antibodies

| | Heavy chain variable domain CDR | | | Light chain variable domain CDR | | |
|---|---|---|---|---|---|---|
| Antibody | CDR1 | CDR2 | CDR3 | CDR1 | CDR2 | CDR3 |
| HL161A | SCVMT | VISGSGGST | TPWWLRS | GGNNIGS | DDSDRP | VRDSSSD |
| | (SEQ ID | YYADSVKG | PFFDY | TSVH | S (SEQ ID | HVI (SEQ |
| | NO: 21) | (SEQ ID NO: | (SEQ ID | (SEQ ID | NO: 25) | ID NO: 26) |
| | | 22) | NO: 23) | NO: 24) | | |
| HL161B | FSYWV | TIYYSGNT | RAGILTGY | GGNNIGS | DDSDRPS | QVWDSSS |
| (HL161BK) | (SEQ ID | YYNPSLKS | LDS (SEQ | KSVH | (SEQ ID | DHVV |
| (HL161BKN) | NO: 27) | (SEQ ID NO: | ID NO: 29) | (SEQ ID | NO: 31) | (SEQ ID |
| | | 28) | | NO: 30) | | NO: 32) |
| HL161C | GCYMH | RINPNSGGT | DYSGWSF | RASQGIS | AASSLQS | QQYDSYP |
| | (SEQ ID | NYAQKFQG | DY (SEQ | NYLA | (SEQ ID | PTF (SEQ |
| | NO: 33) | (SEQ ID NO: | ID NO: 35) | (SEQ ID | NO: 37) | ID NO: 38) |
| | | 34) | | NO: 36) | | |
| HL161D | SYYWG | NIYYSGST | QLSYNWN | GGNNIGS | RDSNRPS | QVWDSST |
| | (SEQ ID | YYNPSLMS | DRLFDY | KNVH | (SEQ ID | VV (SEQ |
| | NO: 39) | (SEQ ID NO: | (SEQ ID | (SEQ ID | NO: 43) | ID NO: 44) |
| | | 40) | NO: 41) | NO: 42) | | |
| HL161ANS | SSVMT | VISGSGGST | TPWWLRS | GGSNIGS | DDSDRPS | VRDSSSD |
| | (SEQ ID | YYADSVKG | PFFDY | TSVH | (SEQ ID | HVI (SEQ |
| | NO: 49) | (SEQ ID NO: | (SEQ ID | (SEQ ID | NO: 25) | ID NO: 26) |
| | | 22) | NO: 23) | NO 50) | | |

In some embodiments, the antibody or antigen-binding fragment comprises three heavy chain CDR amino acid sequences of SEQ TD No: 27 (HCDR1), SEQ TD No: 28 (HCDR2), SEQ ID No: 29 (HCDR3); and three light chain CDR amino acid sequences of SEQ ID No: 30 (LCDR1), SEQ ID No: 31 (LCDR2), SEQ ID No: 32 (LCDR3). In some embodiments, the antibody or antigen-binding fragment comprises three heavy chain CDR amino acid sequences of SEQ ID No: 49 (HCDR1), SEQ ID No: 22 (HCDR2), SEQ ID No: 23 (HCDR3); and three light chain CDR amino acid sequences of SEQ ID No: 50 (LCDR1), SEQ ID No: 25 (LCDR2), SEQ ID No: 26 (LCDR3).

Illustrative heavy and light chain variable regions of anti-FcRn antibodies are set forth in Table 2.

TABLE 2
Amino acid sequences of heavy chain and light chain variable domains of
selected human FcRn antibodies

| | Heavy chain variable domain | Light chain variable domain |
|---|---|---|
| Antibody | amino acid Sequence | amino acid sequence |
| HL161A | EVQLLESGGG LVQPGGSLRL | SYVLTQPPSV SVAPGQTARI |
| | SCAASEFTFG SCVMTWVRQA | TCGGNNIGST SVHWYQQKPG |
| | PGKGLEWVSV ISGSGGSTYY | QAPVLVVHDD SDRPSGIPER |
| | ADSVKGRFTI SRDNSKNTLY | FSGSNSGNTA TLTISRVEAG |
| | LQMNSLRAED TAVYYCAKTP | DEADYYCQVR DSSSDHVIFG |
| | WWLRSPFFDY WGQGTLVTVSS | GGTKLTVLGQ PKAAPSVTL |
| | (SEQ ID NO: 2) | (SEQ ID NO: 12) |
| HL161B | QLLLQESGPG LVKPSETLSL | SYVLTQSPSV SVAPGQTARI |
| | TCTVSGGSLS SSFSYWVWIR | TCGGNNIGSK SVHWYQQKPG |
| | QPPGKGLEWI GTIYYSGNTY | QAPVLVVYDD SDRPSGIPER |
| | YNPSLKSRLT ISVDTSKNHF | FSASNSGNTA TLTISRVEAG |
| | SLNLSSVTAA DTAVYYCARR | DEADYYCQVW DSSSDHVVFG |
| | AGILTGYLDS WGQGTLVTVSS | GGTKLTVLGQ PKAAPSVTL |
| | (SEQ ID NO: 4) | (SEQ ID NO: 14) |
| HL161BK | QLLLQESGPG LVKPSETLSL | SYVLTQSPSV SVAPGQTARI |
| (HL161BKN) | TCTVSGGSLS SSFSYWVWIR | TCGGNNIGSK SVHWYQQKPG |
| | QPPGKGLEWI GTIYYSGNTY | QAPVLVVYDD SDRPSGIPER |
| | YNPSLKSRLT ISVDTSKNHF | FSASNSGNTA TLTISRVEAG |
| | SLKLSSVTAA DTAVYYCARR | DEADYYCQVW DSSSDHVVFG |
| | AGILTGYLDS WGQGTLVTVSS | GGTKLTVLGQ PKAAPSVTL |
| | (SEQ ID NO: 6) | (SEQ ID NO: 16) |
| HL161C | QVQLVQSGAE VKKPGASVKV | DIQMTQSPSS LSASVGDRVT |
| | SCKASGYTFT GCYMHWVRQA | ITCRASQGIS NYLAWFQQKP |
| | PGQGLEWMGR INPNSGGTNY | GKAPKSLIYA ASSLQSGVPS |
| | AQKFQGRVTM TRDTSISTAY | KFSGSGSGTD FTLTISSLQS |
| | MDLSRLRSDD TAVYYCARDY | EDFATYYCQQ YDSYPPTFGG |
| | SGWSFDYWGQ GTLVTVSS | GTKVEIKRTV AAPSVFI (SEQ |
| | (SEQ ID NO: 8) | ID NO: 18) |
| HL161D | QLQLQESGPG LVKPSETLSL | SYELTQPLSV SVALGQTARI |
| | TCTVSGGSIS SSSYYWGWIR | TCGGNNIGSK NVHWYQQKPG |
| | QPPGKGLEWI GNIYYSGSTY | QAPVLVIYRD SNRPSGIPER |
| | YNPSLMSRVT ISVDTSKNQF | FSGSNSGNTA TLTISRAQAG |
| | SLKLSSVTAA DTAVYYCARQ | DEADYYCQVW DSSTVVFGGG |
| | LSYNWNDRLF DYWGQGTLVT | TKLTVLGQPK AAPSVTL |
| | VSS | (SEQ ID NO: 20) |
| | (SEQ ID NO: 10) | |
| HL161ANS | EVQLLESGGG LVQPGGSLRL | SYVLTQPPSV SVAPGQTARI |
| | SCAASEFTFG SSVMTWVRQA | TCGGSNIGST SVHWYQQKPG |
| | PGKGLEWVSV | QAPVLVVHDD |
| | ISGSGGSTYY ADSVKGRFTI | SDRPSGIPER FSGSNSGNTA |
| | SRDNSKNTLY LQMNSLRAED | TLTISRVEAG DEADYYCQVR |
| | TAVYYCAKTP | DSSSDHVIFG |
| | WWLRSPFFDY WGQGTLVTVSS | GGTKLTVLGQ PKAAPSVTL |
| | (SEQ ID NO: 51) | (SEQ ID NO: 52) |

In some embodiments, the antibody or antigen-binding fragment comprises a heavy chain variable region comprising an amino acid sequence that is at least 90%, at least 95%, at least 98%, or least 99 identical to the sequence set forth in SEQ ID No: 6; and a light chain variable region comprising an amino acid sequence that is at least 90%, at least 95%, at least 98%, or least 99 identical to the sequence set forth in SEQ ID No: 16. In some embodiments, the antibody or antigen-binding fragment comprises a heavy chain amino acid comprising an amino acid sequence that is at least 90%, at least 95%, at least 98%, or least 99 identical to the sequence set forth in SEQ ID No: 46; and a light chain amino acid comprising an amino acid sequence that is at least 90%, at least 95%, at least 98%, or least 99 identical to the sequence set forth in SEQ ID No: 48. In some embodiments, the antibody or antigen-binding fragment comprises a heavy chain amino acid comprising an amino acid sequence that is at least 90%, at least 95%, at least 98%, or least 99 identical to the sequence set forth in SEQ ID No: 51; and a light chain amino acid comprising an amino acid sequence that is at least 90%, at least 95%, at least 98%, or least 99 identical to the sequence set forth in SEQ ID No: 52.

In some embodiments, the antibody or antigen-binding fragment comprises a heavy chain variable region amino acid sequence of SEQ ID No: 6; and a light chain variable region amino acid sequence of SEQ ID No: 16. In some embodiments, the antibody or antigen-binding fragment comprises a heavy chain amino acid sequence of SEQ ID No: 46; and a light chain amino acid sequence of SEQ ID No: 48. In some embodiments, the antibody or antigen-binding fragment comprises a heavy chain amino acid sequence of SEQ ID No: 51; and a light chain amino acid sequence of SEQ ID No: 52.

Illustrative heavy chains and light chains of the anti-FcRn antibodies are set forth in Table 3.

TABLE 3
Amino acid sequences of full-length heavy chain and light chain of selected human
FcRn antibodies

| Antibody | | |
|---|---|---|
| name | Heavy chain amino acid sequence | Light chain amino acid sequence |
| HL161BKN | QLLLQESGPG LVKPSETLSL TCTVSGGSLS | SYVLTQSPSV |
| | SSFSYWVWIR QPPGKGLEWI | SVAPGQTARI |
| | GTIYYSGNTY YNPSLKSRLT ISVDTSKNHF | TCGGNNIGSK |
| | SLKLSSVTAA DTAVYYCARR | SVHWYQQKPG |
| | AGILTGYLDS WGQGTLVTVS | QAPVLVVYDD |
| | SASTKGPSVF PLAPSSKSTS | SDRPSGIPER FSASNSGNTA |
| | GGTAALGCLV KDYFPEPVTV | TLTISRVEAG |
| | SWNSGALTSG VHTFPAVLQS | DEADYYCQVW |
| | SGLYSLSSVV TVPSSSLGTQ | DSSSDHVVFG |
| | TYICNVNHKP SNTKVDKRVE | GGTKLTVLGQ |
| | PKSCDKTHTC PPCPAPEAAG GPSVFLFPPK | PKAAPSVTLF |
| | PKDTLMISRT PEVTCVVVDV | PPSSEELQAN KATLVCLISD |
| | SHEDPEVKFN WYVDGVEVHN | FYPGAVTVAW |
| | AKTKPREEQY NSTYRVVSVL | KADSSPVKAG |
| | TVLHQDWLNG KEYKCKVSNK | VETTTPSKQS |
| | ALPAPIEKTI SKAKGQPREP QVYTLPPSRE | NNKYAASSYL |
| | EMTKNQVSLT CLVKGFYPSD | SLTPEQWKSH |
| | IAVEWESNGQ PENNYKTTPP | RSYSCQVTHE |
| | VLDSDGSFFL YSKLTVDKSR | GSTVEKTVAP TECS (SEQ |
| | WQQGNVFSCS VMHEALHNHY | ID NO: 48) |
| | TQKSLSLSPG (SEQ ID NO: 46) | |
| HL161ANS | EVQLLESGGG LVQPGGSLRL | SYVLTQPPSV |
| IgG1 | SCAASEFTFG SSVMTWVRQA | SVAPGQTARI |
| | PGKGLEWVSV ISGSGGSTYY | TCGGSNIGST |
| | ADSVKGRFTI SRDNSKNTLY | SVHWYQQKPG |
| | LQMNSLRAED TAVYYCAKTP | QAPVLVVHDD |
| | WWLRSPFFDY WGQGTLVTVS | SDRPSGIPER FSGSNSGNTA |
| | SASTKGPSVF PLAPSSKSTS | TLTISRVEAG |
| | GGTAALGCLV KDYFPEPVTV | DEADYYCQVR |
| | SWNSGALTSG VHTFPAVLQS | DSSSDHVIFG |
| | SGLYSLSSVV TVPSSSLGTQ | GGTKLTVLGQ |
| | TYICNVNHKP SNTKVDKRVE | PKAAPSVTLF |
| | PKSCDKTHTC PPCPAPEAAG GPSVFLFPPK | PPSSEELQAN KATLVCLISD |
| | PKDTLMISRT PEVTCVVVDV | FYPGAVTVAW |
| | SHEDPEVKFN WYVDGVEVHN | KADSSPVKAG |
| | AKTKPREEQY NSTYRVVSVL | VETTTPSKQS |
| | TVLHQDWLNG KEYKCKVSNK | NNKYAASSYL |
| | ALPAPIEKTI SKAKGQPREP QVYTLPPSRE | SLTPEQWKSH |
| | EMTKNQVSLT CLVKGFYPSD | RSYSCQVTHE |
| | IAVEWESNGQ PENNYKTTPP | GSTVEKTVAP TECS |
| | VLDSDGSFFL YSKLTVDKSR | (SEQ ID NO: 54) |
| | WQQGNVFSCS VMHEALHNHY | |
| | TQKSLSLSPG (SEQ ID NO: 53) | |
| HL161ANS | EVQLLESGGG LVQPGGSLRL | SYVLTQPPSV |
| IgG4 | SCAASEFTFG SSVMTWVRQA | SVAPGQTARI |
| | PGKGLEWVSV ISGSGGSTYY | TCGGSNIGST |
| | ADSVKGRFTI SRDNSKNTLY | SVHWYQQKPG |
| | LQMNSLRAED TAVYYCAKTP | QAPVLVVHDD |
| | WWLRSPFFDY WGQGTLVTVS | SDRPSGIPER FSGSNSGNTA |
| | SASTKGPSVF PLAPCSRSTS ESTAALGCLV | TLTISRVEAG |
| | KDYFPEPVTV SWNSGALTSG | DEADYYCQVR |
| | VHTFPAVLQS SGLYSLSSVV TVPSSSLGTK | DSSSDHVIFG |
| | TYTCNVDHKP SNTKVDKRVE | GGTKLTVLGQ |
| | SKYGPPCPPC PAPEFLGGPS VFLFPPKPKD | PKAAPSVTLF |
| | TLMISRTPEV TCVVVDVSQE | PPSSEELQAN KATLVCLISD |
| | DPEVQFNWYV DGVEVHNAKT | FYPGAVTVAW |
| | KPREEQFNST YRVVSVLTVL | KADSSPVKAG |
| | HQDWLNGKEY KCKVSNKGLP | VETTTPSKQS |
| | SSIEKTISKA KGQPREPQVY TLPPSQEEMT | NNKYAASSYL |
| | KNQVSLTCLV KGFYPSDIAV | SLTPEQWKSH |
| | EWESNGQPEN NYKTTPPVLD | RSYSCQVTHE |
| | SDGSFFLYSR LTVDKSRWQE | GSTVEKTVAP TECS (SEQ |
| | GNVFSCSVMH EALHNHYTQK SLSLSLG | ID NO: 56) |
| | (SEQ ID NO: 55) | |

In some embodiments, the antibody or antigen-binding fragment comprises a heavy chain comprising an amino acid sequence that is at least 9000, at least 9500, at least 98%, or least 99 identical to the sequence set forth in SEQ ID No: 46 53 or 55; and a light chain comprising an amino acid sequence that is at least 90%, at least 9500 at least 98%, or least 99 identical to the sequence set forth in SEQ ID No: 48, 54, or 56. In some embodiments, the antibody or antigen-binding fragment comprises a heavy chain comprising the sequence set forth in SEQ ID No: 46 53 or 55; and a light chain comprising the sequence set forth in SEQ ID No: 48, 54, or 56.

Binding “affinity” refers to the strength of interaction between antibody and antigen at single antigenic sites. Within each antigenic site, the variable region of the antibody “arm” interacts through weak non-covalent forces with the antigen at numerous sites. In general, the more interactions, the higher the affinity.

As used herein, the term “specific,” “specifically binds,” and “binds specifically” refers to a binding reaction between an antibody or an antigen-binding fragment thereof (e.g., an anti-FcRn antibody or an antigen-binding fragment thereof) and a target antigen (e.g., FcRn) in a heterogeneous population of proteins and other biologics. Antibodies can be tested for specificity of binding by comparing binding to an appropriate antigen with binding to an alternate antigen or antigen mixture under a given set of conditions. If the antibody binds to the appropriate antigen with at least 2 times, at least 5 times, or at least 10 times (or more) higher affinity than to the alternate antigen or antigen mixture, then it is considered to be specific.

A “specific antibody” or a “target-specific antibody” is one that only binds the target antigen (e.g., FcRn), but does not bind (or exhibits minimal binding) to other antigens. In some embodiments, an antibody or an antigen-binding fragment thereof that specifically binds the target antigen (e.g., FcRn) has a K_{D} of less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M at pH 6.0 or pH 7.4. In some embodiments, the K_{D} is about 0.01 nM to about 2 nM at pH 6.0 or pH 7.4. In some embodiments, the K_{D} is about 300 pM or less to about 2 nM or less at pH 7.4. In some embodiments, the K_{D} is about 2 nM or less to 900 pM or less at pH 6.0.

As used herein, the term “K_{D}” refers to the equilibrium dissociation constant for antibody-antigen binding, which is obtained from the ratio of k_{d} to kₐ (i.e., k_{d}/kₐ) and is generally expressed as a molar concentration (M). The term “k_{assoc}” or “kₐ” refers to the association rate of a particular antibody-antigen interaction, whereas the term “k_{dis}” or “k_{d}” refers to the dissociation rate of a particular antibody-antigen interaction. The measurement of k_{d} and/or kₐ can be performed at 25° C. or 37° C. K_{D} values for antibodies and antigen-binding fragments can be determined using methods well established in the art (see, e.g., Pollard, Mol. Biol. Cell 21(23):4061-7, 2010). In some embodiments, the K_{D} is measured by direct binding and/or competition binding assays (e.g., surface plasmon resonance and/or competition ELISA). In some embodiments, the K_{D} is measured by surface plasmon resonance (e.g., human FcRn-immobilized surface plasmon resonance). In some embodiments, the K_{D} of an anti-FcRn antibody or antigen-binding fragment disclosed herein is measured by human FcRn-immobilized surface plasmon resonance.

In some embodiments of the therapeutic methods, uses, and compositions disclosed herein, the anti-FcRn antibody or antigen-binding fragment has a K_{D} (dissociation constant) of about 0.01 nM to 2 nM at pH 6.0 and pH 7.4, as determined by, e.g., surface plasmon resonance. In some embodiments, the anti-FcRn antibody or antigen-binding fragment has a K_{D} from about 300 pM or less to about 2 nM or less at pH 7.4 and/or has a K_{D} from about 2 nM or less to about 900 pM or less at pH 6.0, as determined by, e.g., surface plasmon resonance. In some embodiments, the anti-FcRn antibody or antigen-binding fragment binds to the outside of cells and when bound maintains its binding to endosomes. In some embodiments, the anti-FcRn antibody or antigen-binding fragment effectively blocks the binding of one or more autoantibodies to FcRn (e.g., human FcRn), as determined by, e.g., a blocking assay performed using human FcRn-expressing cells and FACS.

As used herein, the term “anti-FcRn antibody” or “antibody that binds specifically to FcRn” refers to any form of an antibody or an antigen-binding fragment thereof that binds specifically to FcRn, e.g., those binding with a K_{D} of less than 2 nM at pH 6.0 or pH 7.4, as determined by, e.g., surface plasmon resonance (e.g., human FcRn-immobilized surface plasmon resonance). The term encompasses monoclonal antibodies (including full-length monoclonal antibodies), polyclonal antibodies, and biologically functional fragments so long as they bind specifically to FcRn.

In some embodiments of the therapeutic methods, uses, and compositions disclosed herein, the anti-FcRn antibody or antigen-binding fragment comprises:
a CDR1 comprising an amino acid sequence that is at least 90% identical to one or more amino acid sequences selected from the group consisting of SEQ ID Nos: 21, 24, 27, 30, 33, 36, 39, 42, 49, and 50;
a CDR2 comprising an amino acid sequence that is at least 90% identical to one or more amino acid sequences selected from the group consisting of SEQ ID Nos: 22, 25, 28, 31, 34, 37, 40, and 43; and
a CDR3 comprising an amino acid sequence that is at least 90% identical to one or more amino acid sequences selected from the group consisting of SEQ ID Nos: 23, 26, 29, 32, 35, 38, 41, and 44.

In some embodiments of the therapeutic methods, uses, and compositions disclosed herein, the anti-FcRn antibody or antigen-binding fragment comprises:
a CDR1 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 21, 24, 27, 30, 33, 36, 39, 42, 49 and 50;
a CDR2 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 22, 25, 28, 31, 34, 37, 40, and 43; and
a CDR3 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 23, 26, 29, 32, 35, 38, 41, and 44.

In some embodiments of the therapeutic methods, uses, and compositions disclosed herein, the anti-FcRn antibody or antigen-binding fragment may comprise one or more amino acid deletions, additions, or substitutions in the amino acid sequences described herein.

In some embodiments of the therapeutic methods, uses, and compositions disclosed herein, the anti-FcRn antibody or antigen-binding fragment may comprise amino acid sequences identical to or having homology with the amino acid sequences described herein. The term “identity” or “homology” refers to a relationship between the sequences of two or more polypeptides, as determined by comparing the sequences. The term “identity” also means the degree of sequence relatedness between the polypeptides, as determined by the number of matches between strings of two or more amino acid residues. The percent “identity” between the two sequences is a function of the number of identical positions shared by the sequences (i.e., percent identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Additionally, or alternatively, the amino acid sequences disclosed herein can further be used as a “query sequence” to perform a search against public databases to, for example, identify related sequences. For example, such searches can be performed using the BLAST program of Altschul et al. (J. Mol. Biol. 215:403-10, 1990).

Two sequences are “substantially identical” if two sequences have a specified percentage of amino acid residues that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 10 amino acids in length, or over a region that is about 20, 50, 200 or more amino acids in length. In some embodiments, the anti-FcRn antibodies and antigen-binding fragments described herein comprise at least one amino acid sequence that is at least 90% identical to a sequence selected from the group consisting of SEQ ID Nos: 2, 4, 6, 8, 10, 12, 14, 16, 18, and 20-48. In some embodiments, the anti-FcRn antibodies and antigen-binding fragments described herein comprise at least one amino acid sequence that is at least 91%, at least 92%, at least 93%, at least 94%, at least 95%, at least 96%, at least 97%, at least 98%, or at least 99% identical to a sequence selected from the group consisting of SEQ ID Nos: 2, 4, 6, 8, 10, 12, 14, 16, 18, and 20-48.

In some embodiments, the antibody or antigen-binding fragment comprises a heavy chain variable region comprising:
a CDR1 comprising an amino acid sequence of SEQ ID No: 21, CDR2 comprising an amino acid sequence of SEQ ID No: 22, and CDR3 comprising an amino acid sequence of SEQ ID No: 23;
a CDR1 comprising an amino acid sequence of SEQ ID No: 27, CDR2 comprising an amino acid sequence of SEQ ID No: 28, and CDR3 comprising an amino acid sequence of SEQ ID No: 29;
a CDR1 comprising an amino acid sequence of SEQ ID No: 33, CDR2 comprising an amino acid sequence of SEQ ID No: 34, and CDR3 comprising an amino acid sequence of SEQ ID No: 35;
a CDR1 comprising an amino acid sequence of SEQ ID No: 39, CDR2 comprising an amino acid sequence of SEQ ID No: 40, and CDR3 comprising an amino acid sequence of SEQ ID No: 41; or
a CDR1 comprising an amino acid sequence of SEQ ID No: 49, CDR2 comprising an amino acid sequence of SEQ ID No: 22, and CDR3 comprising an amino acid sequence of SEQ ID No: 23.

In some embodiments, the antibody or antigen-binding fragment comprises a light chain variable region comprising:
a CDR1 comprising an amino acid sequence of SEQ ID No: 24, CDR2 comprising an amino acid sequence of SEQ ID No: 25, and CDR3 comprising an amino acid sequence of SEQ ID No: 26;
a CDR1 comprising an amino acid sequence of SEQ ID No: 30, CDR2 comprising an amino acid sequence of SEQ ID No: 31, and CDR3 comprising an amino acid sequence of SEQ ID No: 32;
a CDR1 comprising an amino acid sequence of SEQ ID No: 36, CDR2 comprising an amino acid sequence of SEQ ID No: 37, and CDR3 comprising an amino acid sequence of SEQ ID No: 38;
a CDR1 comprising an amino acid sequence of SEQ ID No: 42, CDR2 comprising an amino acid sequence of SEQ ID No: 43, and CDR3 comprising an amino acid sequence of SEQ ID No: 44 or
a CDR1 comprising an amino acid sequence of SEQ ID No: 50, CDR2 comprising an amino acid sequence of SEQ ID No: 25, and CDR3 comprising an amino acid sequence of SEQ ID No: 26.

In some embodiments, the antibody or antigen-binding fragment comprises one or more heavy chain variable regions and one or more light chain variable regions selected from the group consisting of:
a heavy chain variable region comprising CDR1 comprising an amino acid sequence of SEQ ID No: 21 (HCDR1), CDR2 comprising an amino acid sequence of SEQ ID No: 22 (HCDR2), and CDR3 comprising an amino acid sequence of SEQ ID No: 23 (HCDR3); and a light chain variable region comprising CDR1 comprising an amino acid sequence of SEQ ID No: 24 (LCDR1), CDR2 comprising an amino acid sequence of SEQ ID No: 25 (LCDR2), and CDR3 comprising an amino acid sequence of SEQ ID No: 26 (LCDR3);
a heavy chain variable region comprising CDR1 comprising an amino acid sequence of SEQ ID No: 27 (HCDR1), CDR2 comprising an amino acid sequence of SEQ ID No: 28 (HCDR2), and CDR3 comprising an amino acid sequence of SEQ ID No: 29 (HCDR3); and a light chain variable region comprising CDR1 comprising an amino acid sequence of SEQ ID No: 30 (LCDR1), CDR2 comprising an amino acid sequence of SEQ ID No: 31 (LCDR2), and CDR3 comprising an amino acid sequence of SEQ ID No: 32 (LCDR3);
a heavy chain variable region comprising CDR1 comprising an amino acid sequence of SEQ ID No: 33 (HCDR1), CDR2 comprising an amino acid sequence of SEQ ID No: 34 (HCDR2), and CDR3 comprising an amino acid sequence of SEQ ID No: 35 (HCDR3); and a light chain variable region comprising CDR1 comprising an amino acid sequence of SEQ ID No: 36 (LCDR1), CDR2 comprising an amino acid sequence of SEQ ID No: 37 (LCDR2), and CDR3 comprising an amino acid sequence of SEQ ID No: 38 (LCDR3);
a heavy chain variable region comprising CDR1 comprising an amino acid sequence of SEQ ID No: 39 (HCDR1), CDR2 comprising an amino acid sequence of SEQ ID No: 40 (HCDR2), and CDR3 comprising an amino acid sequence of SEQ ID No: 41 (HCDR3); and a light chain variable region comprising CDR1 comprising an amino acid sequence of SEQ ID No: 42 (LCDR1), CDR2 comprising an amino acid sequence of SEQ ID No: 43 (LCDR2), and CDR3 comprising an amino acid sequence of SEQ ID No: 44 (LCDR3); and
a heavy chain variable region comprising CDR1 comprising an amino acid sequence of SEQ ID No: 49 (HCDR1), CDR2 comprising an amino acid sequence of SEQ ID No: 22 (HCDR2), and CDR3 comprising an amino acid sequence of SEQ ID No: 23 (HCDR3); and a light chain variable region comprising CDR1 comprising an amino acid sequence of SEQ ID No: 50 (LCDR1), CDR2 comprising an amino acid sequence of SEQ ID No: 25 (LCDR2), and CDR3 comprising an amino acid sequence of SEQ ID No: 26 (LCDR3).

In some embodiments, the antibody or antigen-binding fragment comprises one or more heavy chain variable regions and/or one or more light chain variable regions comprising one or more amino acid sequences selected from the group consisting of amino acid sequences of SEQ ID Nos: 2, 4, 6, 8, 10, 12, 14, 16, 18, 20, 51, and 52.

In some embodiments, the antibody or antigen-binding fragment comprises heavy chain variable region comprising an amino acid sequence of SEQ ID Nos: 2, 4, 6, 8, 10, or 51 and/or light chain variable region comprising an amino acid sequence of SEQ ID Nos: 12, 14, 16, 18, 20, or 52.

In some embodiments, the antibody or antigen-binding fragment comprises one or more heavy chain variable regions and one or more light chain variable regions selected from the group consisting of:
a heavy chain variable region comprising an amino acid sequence of SEQ ID No: 2 and a light chain variable region comprising an amino acid sequence of SEQ ID No: 12;
a heavy chain variable region comprising an amino acid sequence of SEQ ID No: 4 and a light chain variable region comprising an amino acid sequence of SEQ ID No: 14;
a heavy chain variable region comprising an amino acid sequence of SEQ ID No: 6 and a light chain variable region comprising an amino acid sequence of SEQ ID No: 16;
a heavy chain variable region comprising an amino acid sequence of SEQ ID No: 8 and a light chain variable region comprising an amino acid sequence of SEQ ID No: 18;
a heavy chain variable region comprising an amino acid sequence of SEQ ID No: 10 and a light chain variable region comprising an amino acid sequence of SEQ ID No: 20; and
a heavy chain variable region comprising an amino acid sequence of SEQ ID No: 51 and a light chain variable region comprising an amino acid sequence of SEQ ID No: 52.

The terms “fragment,” “antibody fragment,” and “antigen-binding fragment,” as used herein in reference to an antibody, all refer to one or more fragments of a full-length antibody that retain the ability to specifically bind to the target antigen (e.g., FcRn) and/or provide a function of the full-length antibody (e.g., non-competitive interference with the binding of IgG to FcRn). Antigen-binding fragments can also be present in larger macromolecules, e.g., bispecific, trispecific, and multispecific antibodies. such as diabodies, triabodies and tetrabodies. Examples of antigen-binding fragments include, but are not limited to, single-chain antibodies, Fab fragments, F(ab′)₂ fragments, Fd, scFv, domain antibodies, dual-specific antibodies, minibodies, scap (sterol regulatory binding protein cleavage activating protein), chelating recombinant antibodies, tribodies or bibodies, intrabodies, nanobodies, and small modular immunopharmaceuticals (SMIP).

In some embodiments, the antibody or antigen-binding fragment is a binding-domain immunoglobulin fusion protein, camelized antibody, VHH containing antibody, IgD antibody, IgE antibody, IgM antibody, IgG1 antibody, IgG2 antibody, IgG3 antibody, or IgG4 antibody. In some embodiments, the antibody or antigen-binding fragment comprises derivatives in the antibody constant regions. In some embodiments, the antibody or antigen-binding fragment is a synthetic antibody based on protein scaffolds that have the ability to bind to FcRn.

In some embodiments, an antigen-binding fragment shows the same or similar properties as those of the full-length antibody. Without limitation, an antigen-binding fragment can be produced by any suitable method known in the art. For instance, the various antigen-binding fragments described herein may be produced by enzymatic or chemical modification of full-length antibodies, synthesized de novo using recombinant DNA methodologies (e.g., scFv), or identified using phage display libraries (see, e.g., Pini and Bracci, Curr. Protein Pept. Sci. 1(2):155-69, 2000). Antigen-binding fragments may be screened for utility (e.g., specificity, binding affinity, activity) in the same manner as are full-length antibodies.

In addition, antibodies or antigen-binding fragments having a mutation in the variable and/or constant region may be used in the therapeutic methods, uses, and compositions described herein. Examples of such antibodies or antigen-binding fragments include antibodies having a conservative substitution of an amino acid residue in the variable region and/or constant region. As used herein, the term “conservative substitution” refers to a substitution with another amino acid residue having properties similar to those of the original amino acid residue. For example, lysine, arginine and histidine have similar properties in that they have a basic side-chain, and aspartic acid and glutamic acid have similar properties in that they have an acidic side chain. In addition, glycine, asparagine, glutamine, serine, threonine, tyrosine, cysteine and tryptophan have similar properties in that they have an uncharged polar side-chain, and alanine, valine, leucine, threonine, isoleucine, proline, phenylalanine and methionine have similar properties in that they have a non-polar side-chain. Also, tyrosine, phenylalanine, tryptophan and histidine have similar properties in that they have an aromatic side-chain. Thus, it will be obvious to those skilled in the art that, even when substitution of amino acid residues in groups showing similar properties as described above occurs, it will likely show no significant change in the properties of the antibody or antigen-binding fragment.

In addition, in some embodiments, the antibody or antigen-binding fragment may be conjugated to another substance (e.g., a therapeutic agent or a detectable label). Substances that may be conjugated to, or administered in combination with, an antibody or antigen-binding fragment described herein include but are not limited to therapeutic agents that are generally used for the treatment of Graves' Disease (e.g., a standard-of-care agent, e.g., any one or more of the standard-of-care agents described and/or incorporated by reference herein); substances that are capable of inhibiting the activity of FcRn; and moieties that may be physically associated with the antibody or antigen-binding fragment, e.g., to improve its stabilization and/or retention in circulation, e.g., in blood, serum, lymph, or other tissues. For example, the antibody or antigen-binding fragment can be associated with a polymer, e.g., a non-antigenic polymer such as polyalkylene oxide or polyethylene oxide. Suitable polymers will vary substantially by weight. Polymers having molecular number average weights ranging from about 200 to about 35,000 (or about 1,000 to about 15,000, and 2,000 to about 12,500) can be used. For example, the antibody or antigen-binding fragment can be conjugated to water soluble polymers, e.g., hydrophilic polyvinyl polymers, e.g., polyvinylalcohol and polyvinylpyrrolidone. Non-limiting examples of such polymers include, but are not limited to, polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In various embodiments, the antibody or antigen-binding fragment binds to FcRn with a K_{D} (dissociation constant) of 0.01 nM to 2 nM at pH 6.0 or pH 7.4, as measured by, e.g., surface plasmon resonance.

RVT-1401 (also referred to herein as HL161BKN) is an example of an anti-FcRn antibody. In some embodiments, the antibody or antigen-binding fragment is RVT-1401, or an antigen-binding fragment thereof. In some embodiments, the antibody or antigen-binding fragment comprises the three heavy chain CDR amino acid sequences of RVT-1401 (HCDR1 (SEQ ID No: 27), HCDR2 (SEQ ID No: 28), HCDR3 (SEQ ID No: 29)); and the three light chain CDR amino acid sequences of RVT-1401 (LCDR1 (SEQ ID No: 30), LCDR2 (SEQ ID No: 31), LCDR3 (SEQ ID No: 32)). In some embodiments, the antibody or antigen-binding fragment comprises the heavy chain variable region amino acid sequence of RVT-1401 (SEQ ID No: 6); and the light chain variable region amino acid sequence of RVT-1401 (SEQ ID No: 16). In some embodiments, the antibody or antigen-binding fragment comprises the heavy chain amino acid sequence of RVT-1401 (SEQ ID No: 46); and the light chain amino acid sequence of RVT-1401 (SEQ ID No: 48).

### Pharmaceutical Compositions

Also provided herein are pharmaceutical compositions comprising the anti-FcRn antibody or an antigen-binding fragment thereof formulated together with at least one pharmaceutically acceptable carrier. The compositions may also contain one or more additional therapeutic agents that are suitable for treating for example, Graves' Disease. Methods of formulating pharmaceutical compositions and suitable formulations are known in the art (see, e.g., “Remington's Pharmaceutical Sciences,” Mack Publishing Co., Easton, PA). Appropriate formulation may depend on the route of administration.

As used herein, a “pharmaceutical composition” refers to a preparation of an anti-FcRn antibody or an antigen-binding fragment thereof in addition to other components suitable for administration to a patient, such as a pharmaceutically acceptable carrier and/or excipient. The pharmaceutical compositions provided herein may be suitable for administration in vitro and/or in vivo. In some embodiments, the pharmaceutical compositions provided herein are in such form as to permit administration and subsequently provide the intended biological activity of the active ingredient(s) and/or to achieve a therapeutic effect. The pharmaceutical compositions provided herein preferably contain no additional components which are unacceptably toxic to a subject to which the formulation would be administered.

As used herein, the terms “pharmaceutically acceptable carrier” and “physiologically acceptable carrier,” which may be used interchangeably, refer to a carrier, diluent, or excipient that does not cause significant irritation to a subject and does not abrogate the biological activity and properties of the administered antibody or antigen-binding fragment. Thus, pharmaceutically acceptable carriers should be compatible with the active ingredient such as the antibody or an antigen-binding fragment thereof and may include physiological saline, sterile water, Ringer's solution, buffered saline, dextrose solution, maltodextrin solution, glycerol, ethanol, or a mixture of two or more thereof. Pharmaceutically acceptable carriers may also enhance or stabilize the composition, or can be used to facilitate preparation of the composition. Pharmaceutically acceptable carriers can include other conventional additives, such as antioxidants, buffers, solvents, bacteriostatic agents, dispersion media, coatings, antibacterial and antifungal agents, isotonic and absorption delaying agents, and the like that are physiologically compatible. The carrier may be selected to minimize adverse side effects in the subject, and/or to minimize degradation of the active ingredient(s).

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

In various embodiments of the therapeutic methods, uses, and compositions disclosed herein, the anti-FcRn antibody, antigen-binding fragment, or pharmaceutical composition can be administered by a variety of methods known in the art. The route and/or mode of administration may vary depending upon the desired results. In some embodiments, the antibody, antigen-binding fragment, or pharmaceutical composition is administered by oral, intravenous, intramuscular, intra-arterial, intramedullary, intradural, intracardial, transdermal, subcutaneous, intraperitoneal, gastrointestinal, sublingual, or local routes. In some embodiments, the antibody, antigen-binding fragment, or pharmaceutical composition is administered orally or parenterally. In some embodiments, the antibody, antigen-binding fragment, or pharmaceutical composition is administered parenterally, e.g., intravenously or subcutaneously (e.g., by injection or infusion). In some embodiments, the antibody, antigen-binding fragment, or pharmaceutical composition is administered subcutaneously (e.g., by injection or infusion). In some embodiments, the antibody, antigen-binding fragment, or pharmaceutical composition is administered as one or more subcutaneous injections. In some embodiments, the antibody, antigen-binding fragment, or pharmaceutical composition is administered as a single (i.e., one) subcutaneous injection. In some embodiments, the antibody, antigen-binding fragment, or pharmaceutical composition is administered as two or more (e.g., two) consecutive subcutaneous injections. In some embodiments, the antibody, antigen-binding fragment, or pharmaceutical composition is administered without intravenous administration (e.g., intravenous induction) prior to the one or more subcutaneous injections. In some embodiments, the antibody, antigen-binding fragment, or pharmaceutical composition is delivered via a syringe, a catheter, a pump delivery system, or a stent. In some embodiments, the antibody, antigen-binding fragment, or pharmaceutical composition is delivered via a syringe (e.g., a pre-filled syringe). Depending on the route of administration, the active compound(s), i.e., the anti-FcRn antibody or antigen-binding fragment, may be coated in a material to protect the compound(s) from the action of acids and other natural conditions that may inactivate the compound(s).

An antibody, antigen-binding fragment, or pharmaceutical composition may be formulated as various forms such as a powder, tablet, capsule, liquid, injection, ointment, or syrup, and/or comprised in a single-dosage or multi-dosage container such as a sealed ampoule, vial, or syringe. In some embodiments, the antibody, antigen-binding fragment, or pharmaceutical composition is formulated as an injectable form. In some embodiments, the antibody, antigen-binding fragment, or pharmaceutical composition is formulated as an aqueous solution, suspension, or emulsion, with one or more excipients, diluents, dispersants, surfactants, binders, and/or lubricants. In some embodiments, the antibody, antigen-binding fragment, or pharmaceutical composition is comprised in a syringe (e.g., a pre-filled syringe). In some embodiments, the antibody, antigen-binding fragment, or pharmaceutical composition is comprised in a syringe having and/or compatible with a small gauge needle (e.g., a needle greater than about 25 gauge, greater than about 26 gauge, greater than about 27 gauge, greater than about 28 gauge, greater than about 29 gauge, and/or greater than about 30 gauge).

In some embodiments, the antibody, antigen-binding fragment, or pharmaceutical composition is formulated to achieve stability and/or to prevent or minimize physical and/or chemical degradation prior to administration. Physical instability may involve processes such as denaturation and aggregation, while common chemical degradation pathways include but are not limited to cross-linking, deamidation, isomerization, oxidation, and fragmentation (see, e.g., Wang et al., J. Pharm. Sci. 91(1):1-26, 2007). As used herein, the term “stable” or “stability” when used to describe an antibody or an antigen-binding fragment thereof means that the antibody or antigen-binding fragment remains intact in a manner to retain activity (e.g., binding to FcRn) and/or achieve a therapeutic effect. In some embodiments, the antibody, antigen-binding fragment, or pharmaceutical composition is formulated with one or more pharmaceutically acceptable carriers (e.g., one or more excipients) such that it is stable under standard storage conditions. In some embodiments, the antibody, antigen-binding fragment, or pharmaceutical composition is formulated with one or more pharmaceutically acceptable carriers (e.g., one or more excipients) such that it is stable at high concentrations. In some embodiments, the antibody or antigen-binding fragment can be stably concentrated at formulations up to about 150 mg/mL or above. In some embodiments, the antibody or antigen-binding fragment can be stably concentrated at formulations up to about 170 mg/mL or above. In some embodiments, the antibody or antigen-binding fragment can be stably concentrated at formulations above about 170 mg/mL (e.g., at about 180 mg/mL, at about 200 mg/mL, at about 220 mg/mL, or above). In some embodiments, the stably concentrated formulation (e.g., a formulation comprising up to about 170 mg/mL or above of the antibody or antigen-binding fragment) retains acceptable viscosity for administration via a small gauge needle. In some embodiments, the small gauge needle is greater than about 25 gauge, greater than about 26 gauge, greater than about 27 gauge, greater than about 28 gauge, greater than about 29 gauge, or greater than about 30 gauge.

The anti-FcRn antibody or antigen-binding fragment may be formulated into pharmaceutically acceptable dosage forms by conventional methods known to those of skill in the art. Parenteral compositions, for example, may be formulated in dosage unit form for ease of administration and uniformity of dosage. As used herein, “dosage unit form” refers to physically discrete units suited as unitary dosages for the subjects to be treated; each unit contains a predetermined quantity of active compound calculated to produce the desired therapeutic effect in association with a pharmaceutically acceptable carrier. In some embodiments, the antibody, antigen-binding fragment, or pharmaceutical composition is formulated in dosage unit form. In some embodiments, the antibody, antigen-binding fragment, or pharmaceutical composition is formulated in dosage unit form for subcutaneous administration. In some embodiments, the antibody, antigen-binding fragment, or pharmaceutical composition is formulated in dosage unit form for administration as one or more subcutaneous injections (e.g., one subcutaneous injection or two or more (e.g., two) consecutive subcutaneous injections). In some embodiments, the antibody, antigen-binding fragment, or pharmaceutical composition is formulated in dosage unit form (e.g., as one or more subcutaneous injections) for self-administration by the patient and/or for administration by a treating clinician.

### Kits

In various embodiments, the present disclosure also provides a kit for use in the therapeutic applications described herein. In various embodiments, the present disclosure provides a kit comprising the anti-FcRn antibody or an antigen-binding fragment thereof for use in the treatment of prevention of Graves' Disease. In various embodiments, the kit further comprises one or more additional components, including but not limited to: instructions for use; other agents, e.g., one or more additional therapeutic agents (e.g., one or more standard-of-care agents); devices, containers, or other materials for preparing the antibody or antigen-binding fragment for therapeutic administration; pharmaceutically acceptable carriers (e.g., excipients); and devices, containers, or other materials for administering the antibody or antigen-binding fragment to a patient. In some embodiments, the kit comprises a prefilled syringe comprising a single dose (e.g. a dose provided herein) of the antibody or antigen-binding fragment thereof and instructions for use. Instructions for use can include guidance for therapeutic applications including suggested dosages and/or modes of administration, e.g., in a patient having or suspected of having Graves' Disease. In various embodiments, the kit comprises the anti-FcRn antibody or an antigen-binding fragment thereof and instructions for therapeutic use, e.g., the use of the antibody or antigen-binding fragment to treat or prevent Graves' Disease in a patient. In various embodiments, the kit further contains at least one additional therapeutic agent (e.g., for administering in combination with the antibody or antigen-binding fragment). In various embodiments, the antibody or antigen-binding fragment is formulated as a pharmaceutical composition.

### Methods of Producing Anti-FcRn Antibodies

In some embodiments, the anti-FcRn antibody or antigen-binding fragment is produced by expression and purification using a gene recombination method. In some embodiments, polynucleotide sequences that encode the variable regions of the antibody or antigen-binding fragment are produced by expression in separate host cells or simultaneously in a single host cell. Illustrative polynucleotides encoding the VH and VL and the heavy and light chains of anti-FcRn antibodies are set forth in Table 4 and Table 5, respectively.

TABLE 4
Polynucleotide sequences of heavy chain and light chain variable domains of
selected human FcRn antibodies

| | Heavy chain variable domain | Light chain variable domain |
|---|---|---|
| Antibody | polynucleotide sequence | polynucleotide sequence |
| HL161A | GAAGTGCAGC TGCTGGAATC | TCTTACGTGC TGACCCAGCC |
| | CGGCGGAGGC CTGGTGCAGC | CCCCTCCGTG TCTGTGGCTC |
| | CTGGCGGCTC TCTGAGACTG | CTGGCCAGAC CGCCAGAATC |
| | TCCTGCGCCG CCTCCGAGTT | ACCTGTGGCG GCAACAACAT |
| | CACCTTCGGC AGCTGCGTGA | CGGCTCCACC TCCGTGCACT |
| | TGACCTGGGT CCGACAGGCT | GGTATCAGCA GAAGCCCGGC |
| | CCCGGCAAGG GCCTGGAATG | CAGGCCCCCG TGCTGGTGGT |
| | GGTGTCCGTG ATCTCCGGCT | GCACGACGAC TCCGACCGGC |
| | CCGGCGGCTC CACCTACTAC | CTTCTGGCAT CCCTGAGCGG |
| | GCCGACTCTG TGAAGGGCCG | TTCTCCGGCT CCAACTCCGG |
| | GTTCACCATC TCCCGGGACA | CAACACCGCC ACCCTGACCA |
| | ACTCCAAGAA CACCCTGTAC | TCTCCAGAGT GGAAGCCGGC |
| | CTGCAGATGA ACTCCCTGCG | GACGAGGCCG ACTACTACTG |
| | GGCCGAGGAC ACCGCCGTGT | CCAAGTGCGA GACTCCTCCT |
| | ACTACTGCGC CAAGACCCCC | CCGACCACGT GATCTTCGGC |
| | TGGTGGCTGC GGTCCCCCTT | GGAGGCACCA AGCTGACCGT |
| | CTTCGATTAC TGGGGCCAGG | GCTGGGCCAG CCTAAGGCCG |
| | GCACCCTGGT GACAGTGTCC | CTCCCTCCGT GACCCTG |
| | TCC (SEQ ID NO: 1) | (SEQ ID NO: 11) |
| HL161B | CAACTGTTGC TCCAGGAATC | TCTTACGTGC TGACCCAGTC |
| | CGGTCCTGGT CTTGTAAAGC | CCCCTCCGTG TCCGTGGCTC |
| | CATCTGAGAC TCTCTCCCTT | CTGGCCAGAC CGCCAGAATC |
| | ACCTGTACCG TTAGCGGAGG | ACCTGTGGCG GCAACAACAT |
| | AAGTCTTTCC TCAAGCTTCT | CGGCTCCAAG TCCGTGCACT |
| | CCTACTGGGT GTGGATCAGA | GGTATCAGCA GAAGCCCGGC |
| | CAGCCTCCCG GAAAAGGGTT | CAGGCCCCCG TGCTGGTGGT |
| | GGAGTGGATT GGCACAATAT | GTACGACGAC TCCGACCGGC |
| | ACTACTCCGG CAACACTTAC | CCTCTGGCAT CCCTGAGCGG |
| | TATAACCCCA GCCTGAAGAG | TTCTCCGCCT CCAACTCCGG |
| | CAGGCTGACT ATCTCTGTCG | CAACACCGCC ACCCTGACCA |
| | ACACCAGTAA AAATCACTTT | TCTCCAGAGT GGAAGCCGGC |
| | TCTCTGAATC TGTCTTCAGT | GACGAGGCCG ACTACTACTG |
| | GACCGCAGCC GACACCGCCG | CCAAGTGTGG GACTCCTCCT |
| | TGTATTATTG CGCTCGGCGC | CCGACCACGT GGTGTTCGGC |
| | GCCGGGATTC TGACAGGCTA | GGAGGCACCA AGCTGACCGT |
| | TCTGGATTCA TGGGGCCAGG | GCTGGGCCAG CCTAAGGCCG |
| | GGACATTGGT TACAGTGTCT | CTCCCTCCGT GACCCTG |
| | AGT (SEQ ID NO: 3) | (SEQ ID NO: 13) |
| HL161BK | CAGCTGCTGC TGCAAGAATC | TCTTACGTGC TGACCCAGTC |
| (HL161BKN) | CGGCCCTGGC CTGGTGAAAC | CCCCTCCGTG TCCGTGGCTC |
| | CCTCCGAGAC ACTGTCCCTG | CTGGCCAGAC CGCCAGAATC |
| | ACCTGCACCG TGTCCGGCGG | ACCTGTGGCG GCAACAACAT |
| | CTCCCTGTCC TCCAGCTTCT | CGGCTCCAAG TCCGTGCACT |
| | CCTACTGGGT CTGGATCCGG | GGTATCAGCA GAAGCCCGGC |
| | CAGCCCCCTG GCAAGGGCCT | CAGGCCCCCG TGCTGGTGGT |
| | GGAATGGATC GGCACCATCT | GTACGACGAC TCCGACCGGC |
| | ACTACTCCGG CAACACCTAC | CCTCTGGCAT CCCTGAGCGG |
| | TACAACCCCA GCCTGAAGTC | TTCTCCGCCT CCAACTCCGG |
| | CCGGCTGACC ATCTCCGTGG | CAACACCGCC ACCCTGACCA |
| | ACACCTCCAA GAACCACTTC | TCTCCAGAGT GGAAGCCGGC |
| | AGCCTGAAGC TGTCCTCCGT | GACGAGGCCG ACTACTACTG |
| | GACCGCCGCT GACACCGCCG | CCAAGTGTGG GACTCCTCCT |
| | TGTACTACTG TGCCAGAAGG | CCGACCACGT GGTGTTCGGC |
| | GCCGGCATCC TGACCGGCTA | GGAGGCACCA AGCTGACCGT |
| | CCTGGACTCT TGGGGCCAGG | GCTGGGCCAG CCTAAGGCCG |
| | GCACCCTGGT GACAGTGTCC | CTCCCTCCGT GACCCTG (SEQ |
| | TCC (SEQ ID NO: 5) | ID NO: 15) |
| HL161C | CAGGTGCAGC TCGTGCAGTC | GACATCCAGA TGACCCAGTC |
| | CGGCGCAGAG GTCAAAAAGC | ACCATCATCC CTTTCCGCAT |
| | CTGGTGCATC TGTGAAAGTG | CTGTCGGAGA TAGAGTGACT |
| | AGTTGCAAGG CTAGCGGCTA | ATCACCTGCA GGGCTTCTCA |
| | CACCTTTACC GGATGTTATA | AGGTATTTCC AACTACCTCG |
| | TGCATTGGGT ACGCCAAGCC | CCTGGTTCCA GCAAAAGCCA |
| | CCCGGACAAG GCTTGGAATG | GGTAAAGCCC CAAAGAGCTT |
| | GATGGGGCGT ATCAACCCAA | GATCTACGCC GCTTCTAGTC |
| | ACTCTGGCGG GACTAATTAC | TGCAGAGTGG AGTTCCTAGT |
| | GCCCAGAAGT TTCAGGGAAG | AAGTTCTCCG GCTCTGGCAG |
| | GGTGACTATG ACAAGGGACA | TGGCACAGAT TTTACCTTGA |
| | CATCCATATC CACCGCTTAT | CCATTTCCAG CCTGCAGTCT |
| | ATGGACCTGT CTCGACTGCG | GAGGATTTCG CTACCTACTA |
| | GTCTGATGAT ACAGCCGTTT | TTGTCAGCAG TATGACAGCT |
| | ATTACTGCGC CAGAGACTAC | ATCCCCCCAC ATTTGGGGGG |
| | AGCGGATGGA GCTTCGATTA | GGCACTAAGG TGGAGATAAA |
| | TTGGGGGCAG GGTACTTTGG | ACGGACAGTG GCTGCCCCTT |
| | TCACAGTTTC AAGT (SEQ ID | CTGTCTTTAT T (SEQ ID NO: 17) |
| | NO: 7) | |
| HL161D | CAGCTGCAGT TGCAGGAGTC | AGCTATGAGC TGACCCAGCC |
| | AGGCCCCGGT TTGGTTAAGC | TCTGAGCGTA TCTGTCGCTC |
| | CTTCTGAAAC CCTTTCTCTC | TCGGCCAGAC AGCCAGAATT |
| | ACATGCACAG TATCCGGTGG | ACCTGTGGCG GCAATAACAT |
| | CTCCATCTCC AGTTCAAGTT | AGGATCCAAA AATGTTCACT |
| | ACTACTGGGG ATGGATCCGG | GGTATCAGCA AAAACCTGGC |
| | CAACCCCCAG GAAAAGGGCT | CAAGCTCCCG TGCTCGTGAT |
| | GGAGTGGATT GGCAATATAT | CTACCGGGAC TCTAACCGAC |
| | ATTACTCTGG GTCCACCTAT | CCAGTGGAAT CCCCGAACGC |
| | TACAACCCTT CCCTGATGAG | TTTAGCGGTT CCAACTCTGG |
| | TAGAGTGACC ATCAGCGTGG | AAATACAGCT ACTCTGACTA |
| | ACACAAGCAA AAACCAATTC | TCTCCAGGGC TCAGGCCGGG |
| | AGCCTGAAGC TTTCTAGCGT | GATGAGGCCG ATTACTACTG |
| | GACCGCTGCC GACACAGCTG | CCAGGTGTGG GACTCAAGCA |
| | TCTATTACTG TGCCCGCCAG | CAGTGGTCTT CGGCGGAGGT |
| | CTTAGTTATA ACTGGAATGA | ACCAAGTTGA CTGTTCTTGG |
| | TAGGCTGTTT GATTACTGGG | GCAGCCAAAG GCCGCACCTT |
| | GCCAGGGGAC TCTCGTTACA | CAGTGACCCT G (SEQ ID NO: |
| | GTCAGCAGC (SEQ ID NO: 9) | 19) |

TABLE 5
Polynucleotide sequences of full-length heavy chain and light chain of selected
human FcRn antibodies

| Antibody | Heavy chain polynucleotide sequence | Light chain polynucleotide sequence |
|---|---|---|
| HL161BKN | CAG CTG CTG CTG CAA GAA TCC GGC CCT | TCT TAC GTG CTG ACC |
| | GGC CTG GTG AAA CCC TCC GAG ACA CTG | CAG TCC CCC TCC GTG |
| | TCC CTG ACC TGC ACC GTG TCC GGC GGC | TCC GTG GCT CCT GGC |
| | TCC CTG TCC TCC AGC TTC TCC TAC TGG | CAG ACC GCC AGA |
| | GTC TGG ATC CGG CAG CCC CCT GGC AAG | ATC ACC TGT GGC GGC |
| | GGC CTG GAA TGG ATC GGC ACC ATC TAC | AAC AAC ATC GGC |
| | TAC TCC GGC AAC ACC TAC TAC AAC CCC | TCC AAG TCC GTG CAC |
| | AGC CTG AAG TCC CGG CTG ACC ATC TCC | TGG TAT CAG CAG |
| | GTG GAC ACC TCC AAG AAC CAC TTC AGC | AAG CCC GGC CAG |
| | CTG AAG CTG TCC TCC GTG ACC GCC GCT | GCC CCC GTG CTG GTG |
| | GAC ACC GCC GTG TAC TAC TGT GCC AGA | GTG TAC GAC GAC TCC |
| | AGG GCC GGC ATC CTG ACC GGC TAC CTG | GAC CGG CCC TCT GGC |
| | GAC TCT TGG GGC CAG GGC ACC CTG GTG | ATC CCT GAG CGG TTC |
| | ACA GTG TCC TCC GCC TCC ACC AAG GGC | TCC GCC TCC AAC TCC |
| | CCC TCC GTG TTC CCT CTG GCC CCC TCC | GGC AAC ACC GCC |
| | AGC AAG TCC ACC TCT GGC GGC ACC GCT | ACC CTG ACC ATC TCC |
| | GCC CTG GGC TGT CTG GTG AAA GAC TAC | AGA GTG GAA GCC |
| | TTC CCC GAG CCC GTG ACC GTG TCC TGG | GGC GAC GAG GCC |
| | AAC TCT GGC GCC CTG ACC TCC GGC GTG | GAC TAC TAC TGC CAA |
| | CAC ACC TTC CCT GCC GTG CTG CAG TCC | GTG TGG G?C TCC TCC |
| | TCC GGC CTG TAC TCC CTG TCC AGC GTG | TCC GAC CAC GTG GTG |
| | GTG ACC GTG CCC TCC AGC TCT CTG GGC | TTC GGC GGA GGC |
| | ACC CAG ACC TAC ATC TGC AAC GTG AAC | ACC AAG CTG ACC |
| | CAC AAG CCC TCC AAC ACC AAG GTG GAC | GTG CTG GGC CAG CCT |
| | AAG CGG GTG GAA CCC AAG TCC TGC GAC | AAG GCC GCT CCC TCC |
| | AAG ACC CAC ACC TGT CCC CCC TGT CCT | GTG ACC CTG TTC CCC |
| | GCC CCT GAA GCT GCT GGC GGC CCT AGC | CCA TCC TCC GAG |
| | GTG TTC CTG TTC CCC CCA AAG CCC AAG | GAA CTG CAG GCC |
| | GAC ACC CTG ATG ATC TCC CGG ACC CCC | AAC AAG GCC ACC |
| | GAA GTG ACC TGC GTG GTG GTG GAC GTG | CTG GTC TGC CTG ATC |
| | TCC CAC GAG GAC CCT GAA GTG AAG TTC | TCC GAC TTC TAC CCT |
| | AAT TGG TAC GTG GAC GGC GTG GAA GTG | GGC GCC GTG ACC |
| | CAC AAC GCC AAG ACC AAG CCC AGA GAG | GTG GCC TGG AAG |
| | GAA CAG TAC AAC TCC ACC TAC CGG GTG | GCC GAC AGC TCT CCT |
| | GTG TCC GTG CTG ACC GTG CTG CAC CAG | GTG AAG GCC GGC |
| | GAC TGG CTG AAC GGC AAA GAG TAC AAG | GTG GAA ACC ACC |
| | TGC AAG GTC TCC AAC AAG GCC CTG CCT | ACC CCC TCC AAG |
| | GCC CCC ATC GAA AAG ACC ATC TCC AAG | CAG TCC AAC AAC |
| | GCC AAG GGC CAG CCC CGC GAG CCC CAG | AAA TAC GCC GCC TCC |
| | GTG TAC ACA CTG CCC CCT AGC CGG GAA | TCC TAC CTG TCC CTG |
| | GAG ATG ACC AAG AAC CAG GTG TCC CTG | ACC CCC GAG CAG |
| | ACA TGC CTG GTG AAG GGC TTC TAC CCC | TGG AAG TCC CAC |
| | TCC GAC ATT GCC GTG GAA TGG GAG TCC | CGG TCC TAC AGC TGC |
| | AAC GGC CAG CCC GAG AAC AAC TAC AAG | CAA GTG ACA CAC |
| | ACC ACC CCC CCT GTG CTG GAC TCC GAC | GAG GGC TCC ACC |
| | GGC TCA TTC TTC CTG TAC TCC AAG CTG | GTG GAA AAG ACC |
| | ACC GTG GAC AAG TCC CGG TGG CAG CAG | GTG GCC CCT ACC |
| | GGC AAC GTG TTC TCC TGC TCC GTG ATG | GAG TGC TCC (SEQ ID |
| | CAC GAG GCC CTG CAC AAC CAC TAC ACC | NO: 47) |
| | CAG AAG TCC CTG TCC CTG AGC CCC GGC | |
| | (SEQ ID NO: 45) | |
| HL161ANS | ATGGGCTGGTCCTACATCATCCTGTTTCTGGT | ATGGGCTGGTCCTACA |
| IgG1 | GGCCACCGCCGCCACCGTGCACTCC | TCATCCTGTTTCTGGTG |
| | GAAGTGCAGCTGCTGGAATCCGGCGGAGGCC | GCCACCGCCGCCACCG |
| | TGGTGCAGCCTGGCGGCTCTCTGAGACTGTCC | TGCACTCC |
| | TGCGCCGCCTCCGAGTTCACCTTCGGCAGCAG | TCTTACGTGCTGACCC |
| | CGTGATGACCTGGGTCCGACAGGCTCCCGGC | AGCCCCCCTCCGTGTC |
| | AAGGGCCTGGAATGGGTGTCCGTGATCTCCG | TGTGGCTCCTGGCCAG |
| | GCTCCGGCGGCTCCACCTACTACGCCGACTCT | ACCGCCAGAATCACCT |
| | GTGAAGGGCCGGTTCACCATCTCCCGGGACA | GTGGCGGCTCAAACAT |
| | ACTCCAAGAACACCCTGTACCTGCAGATGAA | CGGCTCCACCTCCGTG |
| | CTCCCTGCGGGCCGAGGACACCGCCGTGTACT | CACTGGTATCAGCAGA |
| | ACTGCGCCAAGACCCCCTGGTGGCTGCGGTCC | AGCCCGGCCAGGCCCC |
| | CCCTTCTTCGATTACTGGGGCCAGGGCACCCT | CGTGCTGGTGGTGCAC |
| | GGTGACAGTGTCCTCCGCCTCCACCAAGGGCC | GACGACTCCGACCGGC |
| | CCTCCGTGTTCCCTCTGGCCCCCTCCAGCAAG | CTTCTGGCATCCCTGA |
| | TCCACCTCTGGCGGCACCGCTGCCCTGGGCTG | GCGGTTCTCCGGCTCC |
| | CCTGGTGAAAGACTACTTCCCCGAGCCCGTGA | AACTCCGGCAACACCG |
| | CCGTGTCCTGGAACTCTGGCGCCCTGACCTCC | CCACCCTGACCATCTC |
| | GGCGTGCACACCTTCCCTGCCGTGCTGCAGTC | CAGAGTGGAAGCCGGC |
| | CTCCGGCCTGTACTCCCTGTCCTCCGTGGTGA | GACGAGGCCGACTACT |
| | CCGTGCCCTCCAGCTCTCTGGGCACCCAGACC | ACTGCCAAGTGCGAGA |
| | TACATCTGCAACGTGAACCACAAGCCCTCCA | CTCCTCCTCCGACCAC |
| | ACACCAAGGTGGACAAGCGGGTGGAACCCAA | GTGATCTTCGGCGGAG |
| | GTCCTGCGACAAGACCCACACCTGTCCCCCCT | GCACCAAGCTGACCGT |
| | GTCCTGCCCCTGAAGCTGCTGGCGGCCCTAGC | GCTGGGCCAGCCTAAG |
| | GTGTTCCTGTTCCCCCCAAAGCCCAAGGACAC | GCCGCTCCCTCCGTGA |
| | CCTGATGATCTCCCGGACCCCCGAAGTGACCT | CCCTGTTCCCCCCATCC |
| | GCGTGGTGGTGGACGTGTCCCACGAGGACCC | TCCGAGGAACTGCAGG |
| | TGAAGTGAAGTTCAATTGGTACGTGGACGGC | CCAACAAGGCCACCCT |
| | GTGGAAGTGCACAACGCCAAGACCAAGCCCA | GGTCTGCCTGATCTCC |
| | GAGAGGAACAGTACAACTCCACCTACCGGGT | GACTTCTACCCTGGCG |
| | GGTGTCTGTGCTGACCGTGCTGCACCAGGACT | CCGTGACCGTGGCCTG |
| | GGCTGAACGGCAAAGAGTACAAGTGCAAGGT | GAAGGCCGACAGCTCT |
| | GTCCAACAAGGCCCTGCCTGCCCCCATCGAA | CCTGTGAAGGCCGGCG |
| | AAGACCATCTCCAAGGCCAAGGGCCAGCCCC | TGGAAACCACCACCCC |
| | GCGAGCCCCAGGTGTACACACTGCCCCCTAG | CTCCAAGCAGTCCAAC |
| | CCGGGAAGAGATGACCAAGAACCAGGTGTCC | AACAAATACGCCGCCT |
| | CTGACCTGTCTGGTGAAAGGCTTCTACCCCTC | CCTCCTACCTGTCCCTG |
| | CGACATTGCCGTGGAATGGGAGTCCAACGGC | ACCCCCGAGCAGTGGA |
| | CAGCCCGAGAACAACTACAAGACCACCCCCC | AGTCCCACCGGTCCTA |
| | CTGTGCTGGACTCCGACGGCTCATTCTTCCTG | CAGCTGCCAAGTGACA |
| | TACTCCAAGCTGACCGTGGACAAGTCCCGGT | CACGAGGGCTCCACCG |
| | GGCAGCAGGGCAACGTGTTCTCCTGCTCCGTG | TGGAAAAGACCGTGGC |
| | ATGCACGAGGCCCTGCACAACCACTACACCC | CCCTACCGAGTGCTCC |
| | AGAAGTCCCTGTCCCTGAGCCCCGGCTGAGTA | TGA |
| | TACTTAATTAACTGGGCCTCATGGGCCTTCCG | (SEQ ID NO: 58) |
| | CTCACTGCCCGCTTTCCAG | |
| | SEQ ID NO: 57) | |
| HL161ANS | ATGGGCTGGTCCTACATCATCCTGTTTCTGGT | ATGGGCTGGTCCTACA |
| IgG4 | GGCTACCGCCGCCACCGTGCATTCT | TCATCCTGTTTCTGGTG |
| | GAAGTTCAGCTGTTGGAGTCTGGCGGCGGATT | GCCACCGCCGCCACCG |
| | GGTTCAACCTGGCGGATCTCTGAGACTGTCTT | TGCACTCC |
| | GCGCCGCCTCTGAGTTCACCTTCGGCTCCTCT | TCTTACGTGCTGACCC |
| | GTGATGACCTGGGTCCGACAGGCTCCTGGCA | AGCCCCCCTCCGTGTC |
| | AAGGACTGGAATGGGTGTCCGTGATCTCTGG | TGTGGCTCCTGGCCAG |
| | CTCTGGCGGCTCTACCTACTACGCCGATTCTG | ACCGCCAGAATCACCT |
| | TGAAGGGCAGATTCACCATCAGCCGGGACAA | GTGGCGGCTCAAACAT |
| | CTCCAAGAACACCCTGTACCTGCAGATGAACT | CGGCTCCACCTCCGTG |
| | CCCTGAGAGCCGAGGACACCGCCGTGTACTA | CACTGGTATCAGCAGA |
| | TTGTGCTAAGACCCCTTGGTGGCTGCGGTCCC | AGCCCGGCCAGGCCCC |
| | CTTTCTTTGATTATTGGGGCCAGGGCACCCTG | CGTGCTGGTGGTGCAC |
| | GTCACCGTGTCCTCTGCTTCTACAAAGGGCCC | GACGACTCCGACCGGC |
| | CTCTGTGTTCCCTCTGGCTCCTTGCTCTAGATC | CTTCTGGCATCCCTGA |
| | CACCTCCGAGTCTACCGCTGCTCTGGGCTGTC | GCGGTTCTCCGGCTCC |
| | TGGTCAAGGACTACTTTCCTGAGCCTGTGACC | AACTCCGGCAACACCG |
| | GTGTCTTGGAACTCTGGTGCTCTGACCTCCGG | CCACCCTGACCATCTC |
| | CGTGCACACATTTCCAGCTGTGCTGCAGTCCT | CAGAGTGGAAGCCGGC |
| | CCGGCCTGTACTCTCTGTCCTCTGTCGTGACC | GACGAGGCCGACTACT |
| | GTGCCTTCTAGCTCTCTGGGCACCAAGACCTA | ACTGCCAAGTGCGAGA |
| | CACCTGTAACGTGGACCACAAGCCTTCCAAC | CTCCTCCTCCGACCAC |
| | ACCAAGGTGGACAAGCGCGTGGAATCTAAGT | GTGATCTTCGGCGGAG |
| | ACGGCCCTCCTTGTCCTCCATGTCCTGCTCCA | GCACCAAGCTGACCGT |
| | GAATTCCTCGGCGGACCCTCCGTGTTCCTGTT | GCTGGGCCAGCCTAAG |
| | TCCTCCAAAGCCTAAGGACACCCTGATGATCT | GCCGCTCCCTCCGTGA |
| | CTCGGACCCCTGAAGTGACCTGCGTGGTGGTG | CCCTGTTCCCCCCATCC |
| | GATGTGTCTCAAGAGGACCCCGAGGTGCAGT | TCCGAGGAACTGCAGG |
| | TCAATTGGTACGTGGACGGCGTGGAAGTGCA | CCAACAAGGCCACCCT |
| | CAACGCCAAGACCAAGCCTAGAGAGGAACAG | GGTCTGCCTGATCTCC |
| | TTCAACTCCACCTACAGAGTGGTGTCTGTGCT | GACTTCTACCCTGGCG |
| | GACCGTGCTGCACCAGGATTGGCTGAACGGC | CCGTGACCGTGGCCTG |
| | AAAGAGTACAAGTGCAAGGTGTCCAACAAGG | GAAGGCCGACAGCTCT |
| | GCCTGCCTTCCAGCATCGAAAAGACCATCTCC | CCTGTGAAGGCCGGCG |
| | AAGGCTAAGGGCCAGCCTCGGGAACCTCAGG | TGGAAACCACCACCCC |
| | TTTACACCCTGCCTCCAAGCCAAGAGGAAAT | CTCCAAGCAGTCCAAC |
| | GACCAAGAACCAGGTGTCCCTGACCTGCCTC | AACAAATACGCCGCCT |
| | GTGAAGGGCTTCTACCCTTCCGACATTGCCGT | CCTCCTACCTGTCCCTG |
| | GGAATGGGAGTCTAACGGCCAGCCAGAGAAC | ACCCCCGAGCAGTGGA |
| | AACTACAAGACAACCCCTCCTGTGCTGGACTC | AGTCCCACCGGTCCTA |
| | CGACGGCTCTTTCTTCCTGTATTCTCGCCTGAC | CAGCTGCCAAGTGACA |
| | CGTGGACAAGTCTCGGTGGCAAGAGGGCAAC | CACGAGGGCTCCACCG |
| | GTGTTCTCCTGTTCTGTGATGCACGAGGCCCT | TGGAAAAGACCGTGGC |
| | GCACAACCACTACACCCAGAAGTCCCTGTCTC | CCCTACCGAGTGCTCC |
| | TGTCC CTG GGC TAATGA | TGA |
| | (SEQ ID NO: 59) | (SEQ ID NO: 60) |

As used herein, the term “recombinant vector” refers to an expression vector capable of expressing a protein of interest in a suitable host cell. The term encompasses a DNA construct including essential regulatory elements operably linked to express a nucleic acid insert.

As used herein, the term “operably linked” refers to a nucleic acid expression control sequence functionally linked to a nucleic acid sequence encoding a protein of interest so as to execute general functions. Operable linkage with the recombinant vector can be performed using a gene recombination technique well known in the art, and site-specific DNA cleavage and ligation can be easily performed using enzymes generally known in the art.

A suitable expression vector may include expression regulatory elements such as a promoter, an operator, an initiation codon, a stop codon, a polyadenylation signal, and an enhancer, as well as a signal sequence for membrane targeting or secretion. The initiation and stop codons are generally considered as part of a nucleotide sequence encoding the immunogenic target protein, and are necessary to be functional in an individual to whom a genetic construct has been administered, and must be in frame with the coding sequence. Promoters may generally be constitutive or inducible. Prokaryotic promoters include, but are not limited to, lac, tac, T3 and T7 promoters. Eukaryotic promoters include, but are not limited to, simian virus 40 (SV40) promoter, mouse mammary tumor virus (MMTV) promoter, human immunodeficiency virus (HIV) promoter such as the HIV Long Terminal Repeat (LTR) promoter, moloney virus promoter, cytomegalovirus (CMV) promoter, epstein barr virus (EBV) promoter, rous sarcoma virus (RSV) promoter, as well as promoters from human genes such as human β-actin, human hemoglobin, human muscle creatine, and human metallothionein. The expression vector may include a selectable marker that allows selection of host cells containing the vector. Genes coding for products that confer selectable phenotypes, such as resistance to drugs, a nutrient requirement, or resistance to cytotoxic agents or expression of surface proteins, may be used as general selectable markers. Since only cells expressing a selectable marker survive in the environment treated with a selective agent, transformed cells can be selected. Also, a replicable expression vector may include a replication origin, a specific nucleic acid sequence that initiates replication. Recombinant expression vectors that may be used include various vectors such as plasmids, viruses, and cosmids. The kind of recombinant vector is not limited, and the recombinant vector could function to express a desired gene and produce a desired protein in various host cells such as prokaryotic and eukaryotic cells. In some embodiments, a vector that can produce a large amount of a foreign protein similar to a natural protein while having strong expression ability with a promoter showing strong activity is used.

A variety of expression host/vector combinations may be used to express the anti-FcRn antibody or an antigen-binding fragment thereof. For example, expression vectors suitable for the eukaryotic host include, but are not limited to, SV40, bovine papillomavirus, adenovirus, adeno-associated virus, cytomegalovirus, and retrovirus. Expression vectors that may be used for bacterial hosts include bacterial plasmids such as pET, pRSET, pBluescript, pGEX2T, pUC, col E1, pCR1, pBR322, pMB9 and derivatives thereof, a plasmid such as RP4 having a wider host range, phage DNA represented as various phage lambda derivatives such as gt10, gt11 and NM989, and other DNA phages such as M13 and filamentous single-stranded DNA phage. Expression vectors useful in yeast cells include 2 μm plasmid and derivatives thereof. A vector useful in insect cells is pVL941.

In some embodiments, the recombinant vector is introduced into a host cell to form a transformant. Host cells suitable for use include prokaryotic cells such as E. coli, Bacillus subtilis, Streptomyces sp., Pseudomonas sp., Proteus mirabilis and Staphylococcus sp., fungi such as Aspergillus sp., yeasts such as Pichia pastoris, Saccharomyces cerevisiae, Schizosaccharomyces sp., and Neurospora crassa, and eukaryotic cells such as lower eukaryotic cells, and higher other eukaryotic cells such as insect cells.

In some embodiments, host cells are derived from plants or animals (e.g., mammals), and examples thereof include, but are not limited to, monkey kidney cells (COS7), NSO cells, SP2/0, Chinese hamster ovary (CHO) cells, W138, baby hamster kidney (BHK) cells, MDCK, myeloma cells, HuT 78 cells and HEK293 cells. In some embodiments, CHO cells are used.

Transfection or transformation into a host cell may include any method by which nucleic acids can be introduced into organisms, cells, tissues or organs, and, as known in the art, may be performed using a suitable standard technique selected according to the kind of host cell. Methods include, but are not limited to, electroporation, protoplast fusion, calcium phosphate (CaPO₄) precipitation, calcium chloride (CaCl₂)) precipitation, agitation with silicon carbide fiber, and agrobacterium-, PEG-, dextran sulfate-, lipofectamine- and desiccation/inhibition-mediated transformation.

The anti-FcRn antibody or antigen-binding fragment can be produced in large amounts by culturing the transformant comprising the recombinant vector in nutrient medium, and the medium and culture conditions that are used can be selected depending on the kind of host cell. During culture, conditions, including temperature, the pH of medium, and culture time, can be controlled so as to be suitable for the growth of cells and the mass production of protein. The antibody or antigen-binding fragment produced by the recombination method as described herein can be collected from the medium or cell lysate and can be isolated and purified by conventional biochemical isolation techniques (Sambrook et al., Molecular Cloning: A Laboratory Manual, 2nd Ed., Cold Spring Harbor Laboratory Press (1989); Deuscher, Guide to Protein Purification Methods Enzymology, Vol. 182. Academic Press. Inc., San Diego, CA (1990)). These techniques include, but are not limited to, electrophoresis, centrifugation, gel filtration, precipitation, dialysis, chromatography (e.g., ion exchange chromatography, affinity chromatography, immunosorbent chromatography, size exclusion chromatography, etc.), isoelectric point focusing, and various modifications and combinations thereof. In some embodiments, the antibody or antigen-binding fragment is isolated and purified using protein A.

### EXAMPLES

Hereinafter, the present disclosure will be described in further detail with reference to examples. It will be obvious to a person having ordinary skill in the art that these examples are illustrative purposes only and are not to be construed to limit the scope of the present disclosure.

### Example 1: Clinical Study of Batoclimab Treatment in Graves' Disease

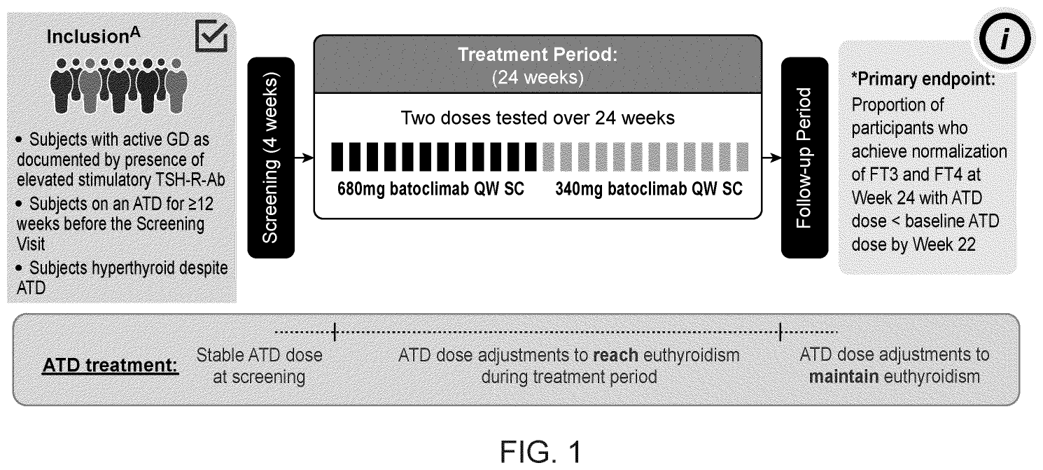

This Example describes a Phase 2, open-label trial of Batoclimab treatment in Graves' Disease. A schematic of the clinical trial is shown in FIG. 1. Subjects are screened and enrolled if they are receiving a stable dose of anti-thyroid medications (ATD). Subjects receive 680 mg Batoclimab weekly by subcutaneous injection for 12 weeks, followed by 340 mg Batoclimab weekly by subcutaneous injection for 12 weeks. ATD dose adjustments may be made to reach and maintain euthyroidism during the treatment period.

The primary endpoint of the trial is the proportion of participants who achieve normalization of free triiodothyronine (FT3) and free thyroxine (FT4) or have FT3 and/or FT4 below the lower limit of normal (LLN) at Week 24 without increase in ATD dose (compared to the dose that was administered at the beginning of the trial, the Baseline ATD dose).

Secondary endpoints include the percentage of participants who achieve normalization of FT3 and FT4 at Week 24 with less than 50% of the Baseline ATD dose by Week 24 and the percentage of participants who are off ATD treatment and achieve normalization of FT3 and FT4, or have FT3 and/or FT4 below the LLN at Week 24.

### Inclusion Criteria:

Have serologically confirmed GD as documented by presence of elevated stimulatory thyrotropin receptor antibody (TSH-R-Ab) level (i.e., >specimen-to-reference ratio of 140%) at the Screening Visit.
Have active hyperthyroidism due to GD with the following laboratory values at the Screening Visit:
   TSH<LLN
   FT3>upper limit of normal (ULN) and ≤5×ULN (i.e., 5 times ULN)
   FT4>ULN and ≤5*ULN
   Note: Participants who have T3 thyrotoxicosis (i.e TSH<ULN, FT3>ULN and ≤5×ULN, but FT4 within normal range) at the Screening Visit may be enrolled, if they have serologically confirmed GD.
Have been on an ATD for ≥12 weeks before the Screening Visit. Had starting total daily ATD dose of >=20 mg of methimazole or carbimazole, or ≥200 mg of propylthiouracil and are currently treated with a total daily dose of ≥10 mg of methimazole or carbimazole, or ≥100 mg of propylthiouracil.
Are willing and capable of giving written informed consent, which includes being able to comply with all aspects of the study treatment and testing schedule.

### Exclusion Criteria:

History of hyperthyroidism not caused by GD (e.g., toxic adenoma or toxic multinodular goiter), and/or history or presence of thyroid storm.
History of treatment with radioactive iodine thyroid surgery.
Total immunoglobulin G (IgG) level<6 grams per liter (g/L) at the Screening Visit.
Albumin level<3.5 grams per deciliter (g/dL) (<35 g/L) at the Screening Visit.
Absolute neutrophil count<1000 cells per cubic millimeter (cells/mm³) at the Screening Visit.

### Example 2: HL161-ANS for the Treatment of Graves' Disease

A study of HL161ANS in patients with Graves' Disease is to be designed in accordance with the study provided in example 1. Subjects receive any of the dosing regiments disclosed herein, for example, 300 mg HL161ANS subcutaneously once weekly for 12 weeks or 600 mg HL161ANS subcutaneously once weekly for 12 weeks. Subjects may also receive 300 mg HL161ANS subcutaneously once weekly for 12 weeks followed by 600 mg HL161ANS subcutaneously once weekly for 12 weeks.

Primary endpoints to be measured include the proportion of participants who achieve normalization of free triiodothyronine (FT3) and free thyroxine (FT4) or have FT3 and/or FT4 below the lower limit of normal (LLN) at Week 24 without increase in ATD dose (compared to the dose that was administered at the beginning of the trial, the Baseline ATD dose). Secondary endpoints include the percentage of participants who achieve normalization of FT3 and FT4 at Week 24 with less than 50% of the Baseline ATD dose by Week 24 and the percentage of participants who are off ATD treatment and achieve normalization of FT3 and FT4, or have FT3 and/or FT4 below the LLN at Week 24.

The inclusion criteria to be used are the same as those disclosed in Example 1.

Although the present disclosure has been described in detail with reference to the specific features, it will be apparent to those skilled in the art that this description is only for purposes of illustration and does not limit the scope of the present disclosure. Thus, the substantial scope of the present disclosure will be defined by the appended claims and equivalents thereof.

1. A method of treating Graves' Disease in a patient in need thereof, comprising administering to the patient a first therapeutically effective amount of an anti-FcRn antibody or an antigen-binding fragment thereof, wherein the antibody or antigen-binding fragment comprises
(a) a heavy chain variable region comprising a HCDR1 comprising the amino acid sequence set forth in SEQ ID No: 27, a HCDR2 comprising the amino acid sequence set forth in SEQ ID No: 28, and a HCDR3 comprising the amino acid sequence set forth in SEQ ID No: 29; and a light chain variable region comprising an LCDR1 comprising the amino acid sequence set forth in SEQ ID No: 30, an LCDR2 comprising the amino acid sequence of SEQ ID No: 31, and an LCDR3 comprising the amino acid sequence set forth in SEQ ID No: 32; or
(b) a heavy chain variable region comprising a HCDR1 comprising the amino acid sequence set forth in SEQ ID No: 49, a HCDR2 comprising the amino acid sequence set forth in SEQ ID No: 22, and a HCDR3 comprising the amino acid sequence set forth in SEQ ID No: 23; and a light chain variable region comprising an LCDR1 comprising the amino acid sequence set forth in SEQ ID No: 50, an LCDR2 comprising the amino acid sequence of SEQ ID No: 25, and an LCDR3 comprising the amino acid sequence set forth in SEQ ID No: 26.

2. The method of claim 1, wherein the antibody or antigen-binding fragment comprises
(a) a heavy chain variable region comprising an amino acid sequence that is at least 90%, at least 95%, at least 98%, or least 99% identical to the sequence set forth in SEQ ID No: 6, and a light chain variable region comprising an amino acid sequence that is at least 90%, at least 95%, at least 98%, or least 99% identical to the sequence set forth in SEQ ID No: 16; or
(b) a heavy chain variable region comprising an amino acid sequence that is at least 90%, at least 95%, at least 98%, or least 99% identical to the sequence set forth in SEQ ID No: 51, and a light chain variable region comprising an amino acid sequence that is at least 90%, at least 95%, at least 98%, or least 99% identical to the sequence set forth in SEQ ID No: 52.

3. The method of claim 1, wherein the antibody or antigen-binding fragment comprises
(a) a heavy chain variable region comprising the sequence set forth in SEQ ID No: 6, and a light chain variable region comprising the sequence set forth in SEQ ID No: 16; or
(b) a heavy chain variable region comprising the sequence set forth in SEQ ID No: 51, and a light chain variable region comprising the sequence set forth in SEQ ID No: 52.

4. The method of any one of claims 1-3, wherein the antibody or antigen-binding fragment binds to FcRn with a K_{D} (dissociation constant) of 0.01 nM to 2 nM at pH 6.0 or pH 7.4.

5. The method of claim 4, wherein the K_{D} is measured by surface plasmon resonance (SPR).

6. The method of any one of claims 1-5, wherein the antibody or antigen-binding fragment is administered subcutaneously.

7. The method of any one of claims 1-6, wherein the antibody or antigen-binding fragment is administered once weekly.

8. The method of any one of claims 1-7, wherein the first therapeutically effective amount of the antibody or antigen-binding fragment is 500 mg to 700 mg.

9. The method of claim 8, wherein the first therapeutically effective amount of the antibody or antigen-binding fragment is 680 mg.

10. The method of any one of claims 1-9, wherein the first effective amount of the antibody or antigen-binding fragment is administered for 12 weeks.

11. The method of any one of claims 1-10, further comprising administering a second therapeutically effective amount of the antibody or antigen-binding fragment to the subject.

12. The method of claim 11, wherein the second therapeutically effective amount is 340 mg.

13. The method of claim 11 or 12, wherein the second therapeutically effective amount of the antibody or antigen-binding fragment is administered for 12 weeks.

14. The method of any one of claims 1-13, wherein the antibody, antigen-binding fragment, or pharmaceutical composition is administered in combination with at least one additional therapeutic agent.

15. The method of any one of claims 1-14, wherein the patient is receiving one or more antithyroid drug (ATD).

16. The method of claim 15, wherein the amount of ATD the patient is receiving is reduced after administration of the first or second effective amount of the antibody or antigen-binding fragment thereof.

17. A method of treating Graves' Disease in a patient in need thereof, comprising administering to the patient a first therapeutically effective amount of an anti-FcRn antibody or an antigen-binding fragment thereof, wherein the antibody or antigen-binding fragment comprises:
(a) a heavy chain variable region comprising a HCDR1 comprising the amino acid sequence set forth in SEQ ID No: 27, a HCDR2 comprising the amino acid sequence set forth in SEQ ID No: 28, and a HCDR3 comprising the amino acid sequence set forth in SEQ ID No: 29; and a light chain variable region comprising an LCDR1 comprising the amino acid sequence set forth in SEQ ID No: 30, an LCDR2 comprising the amino acid sequence of SEQ ID No: 31, and an LCDR3 comprising the amino acid sequence set forth in SEQ ID No: 32; or
(b) a heavy chain variable region comprising a HCDR1 comprising the amino acid sequence set forth in SEQ ID No: 49, a HCDR2 comprising the amino acid sequence set forth in SEQ ID No: 22, and a HCDR3 comprising the amino acid sequence set forth in SEQ ID No: 23; and a light chain variable region comprising an LCDR1 comprising the amino acid sequence set forth in SEQ ID No: 50, an LCDR2 comprising the amino acid sequence of SEQ ID No: 25, and an LCDR3 comprising the amino acid sequence set forth in SEQ ID No: 26; and
wherein the first therapeutically effective amount of the antibody or antigen-binding fragment is 680 mg administered subcutaneously once weekly, optionally wherein the administering is for about 12 weeks or more.

18. The method of claim 17, further comprising administering a second therapeutically effective amount of the antibody or antigen-binding fragment to the patient, wherein the second therapeutically effective amount is 340 mg administered subcutaneously once weekly, optionally wherein the administering is for about 12 weeks or more.

19. The method of any one of claims 1-18, wherein the patient is human.


SEQUENCE LISTING (60 sequences)

SEQ ID NO: 1 - DNA, 363 bp, synthetic construct
  source 1..363: mol_type = other DNA
gaagtgcagctgctggaatccggcggaggcctggtgcagcctggcggctctctgagactgtcctgcgccgcctccgagttcaccttcggcagctgcgtgatgacctgggtccgacaggctcccggcaagggcctggaatgggtgtccgtgatctccggctccggcggctccacctactacgccgactctgtgaagggccggttcaccatctcccgggacaactccaagaacaccctgtacctgcagatgaactccctgcgggccgaggacaccgccgtgtactactgcgccaagaccccctggtggctgcggtcccccttcttcgattactggggccagggcaccctggtgacagtgtcctcc

SEQ ID NO: 2 - protein, 121 aa, synthetic construct
EVQLLESGGGLVQPGGSLRLSCAASEFTFGSCVMTWVRQAPGKGLEWVSVISGSGGSTYYADSVKGRFTISRDNSKNTLYLQMNSLRAEDTAVYYCAKTPWWLRSPFFDYWGQGTLVTVSS

SEQ ID NO: 3 - DNA, 363 bp, synthetic construct
  source 1..363: mol_type = other DNA
caactgttgctccaggaatccggtcctggtcttgtaaagccatctgagactctctcccttacctgtaccgttagcggaggaagtctttcctcaagcttctcctactgggtgtggatcagacagcctcccggaaaagggttggagtggattggcacaatatactactccggcaacacttactataaccccagcctgaagagcaggctgactatctctgtcgacaccagtaaaaatcacttttctctgaatctgtcttcagtgaccgcagccgacaccgccgtgtattattgcgctcggcgcgccgggattctgacaggctatctggattcatggggccaggggacattggttacagtgtctagt

SEQ ID NO: 4 - protein, 121 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLNLSSVTAADTAVYYCARRAGILTGYLDSWGQGTLVTVSS

SEQ ID NO: 5 - DNA, 363 bp, synthetic construct
  source 1..363: mol_type = other DNA
cagctgctgctgcaagaatccggccctggcctggtgaaaccctccgagacactgtccctgacctgcaccgtgtccggcggctccctgtcctccagcttctcctactgggtctggatccggcagccccctggcaagggcctggaatggatcggcaccatctactactccggcaacacctactacaaccccagcctgaagtcccggctgaccatctccgtggacacctccaagaaccacttcagcctgaagctgtcctccgtgaccgccgctgacaccgccgtgtactactgtgccagaagggccggcatcctgaccggctacctggactcttggggccagggcaccctggtgacagtgtcctcc

SEQ ID NO: 6 - protein, 121 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSS

SEQ ID NO: 7 - DNA, 354 bp, synthetic construct
  source 1..354: mol_type = other DNA
caggtgcagctcgtgcagtccggcgcagaggtcaaaaagcctggtgcatctgtgaaagtgagttgcaaggctagcggctacacctttaccggatgttatatgcattgggtacgccaagcccccggacaaggcttggaatggatggggcgtatcaacccaaactctggcgggactaattacgcccagaagtttcagggaagggtgactatgacaagggacacatccatatccaccgcttatatggacctgtctcgactgcggtctgatgatacagccgtttattactgcgccagagactacagcggatggagcttcgattattgggggcagggtactttggtcacagtttcaagt

SEQ ID NO: 8 - protein, 118 aa, synthetic construct
QVQLVQSGAEVKKPGASVKVSCKASGYTFTGCYMHWVRQAPGQGLEWMGRINPNSGGTNYAQKFQGRVTMTRDTSISTAYMDLSRLRSDDTAVYYCARDYSGWSFDYWGQGTLVTVSS

SEQ ID NO: 9 - DNA, 369 bp, synthetic construct
  source 1..369: mol_type = other DNA
cagctgcagttgcaggagtcaggccccggtttggttaagccttctgaaaccctttctctcacatgcacagtatccggtggctccatctccagttcaagttactactggggatggatccggcaacccccaggaaaagggctggagtggattggcaatatatattactctgggtccacctattacaacccttccctgatgagtagagtgaccatcagcgtggacacaagcaaaaaccaattcagcctgaagctttctagcgtgaccgctgccgacacagctgtctattactgtgcccgccagcttagttataactggaatgataggctgtttgattactggggccaggggactctcgttacagtcagcagc

SEQ ID NO: 10 - protein, 123 aa, synthetic construct
QLQLQESGPGLVKPSETLSLTCTVSGGSISSSSYYWGWIRQPPGKGLEWIGNIYYSGSTYYNPSLMSRVTISVDTSKNQFSLKLSSVTAADTAVYYCARQLSYNWNDRLFDYWGQGTLVTVSS

SEQ ID NO: 11 - DNA, 357 bp, synthetic construct
  source 1..357: mol_type = other DNA
tcttacgtgctgacccagcccccctccgtgtctgtggctcctggccagaccgccagaatcacctgtggcggcaacaacatcggctccacctccgtgcactggtatcagcagaagcccggccaggcccccgtgctggtggtgcacgacgactccgaccggccttctggcatccctgagcggttctccggctccaactccggcaacaccgccaccctgaccatctccagagtggaagccggcgacgaggccgactactactgccaagtgcgagactcctcctccgaccacgtgatcttcggcggaggcaccaagctgaccgtgctgggccagcctaaggccgctccctccgtgaccctg

SEQ ID NO: 12 - protein, 119 aa, synthetic construct
SYVLTQPPSVSVAPGQTARITCGGNNIGSTSVHWYQQKPGQAPVLVVHDDSDRPSGIPERFSGSNSGNTATLTISRVEAGDEADYYCQVRDSSSDHVIFGGGTKLTVLGQPKAAPSVTL

SEQ ID NO: 13 - DNA, 357 bp, synthetic construct
  source 1..357: mol_type = other DNA
tcttacgtgctgacccagtccccctccgtgtccgtggctcctggccagaccgccagaatcacctgtggcggcaacaacatcggctccaagtccgtgcactggtatcagcagaagcccggccaggcccccgtgctggtggtgtacgacgactccgaccggccctctggcatccctgagcggttctccgcctccaactccggcaacaccgccaccctgaccatctccagagtggaagccggcgacgaggccgactactactgccaagtgtgggactcctcctccgaccacgtggtgttcggcggaggcaccaagctgaccgtgctgggccagcctaaggccgctccctccgtgaccctg

SEQ ID NO: 14 - protein, 119 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTL

SEQ ID NO: 15 - DNA, 357 bp, synthetic construct; identical to SEQ ID NO: 13
  source 1..357: mol_type = other DNA

SEQ ID NO: 16 - protein, 119 aa, synthetic construct; identical to SEQ ID NO: 14

SEQ ID NO: 17 - DNA, 351 bp, synthetic construct
  source 1..351: mol_type = other DNA
gacatccagatgacccagtcaccatcatccctttccgcatctgtcggagatagagtgactatcacctgcagggcttctcaaggtatttccaactacctcgcctggttccagcaaaagccaggtaaagccccaaagagcttgatctacgccgcttctagtctgcagagtggagttcctagtaagttctccggctctggcagtggcacagattttaccttgaccatttccagcctgcagtctgaggatttcgctacctactattgtcagcagtatgacagctatccccccacatttggggggggcactaaggtggagataaaacggacagtggctgccccttctgtctttatt

SEQ ID NO: 18 - protein, 117 aa, synthetic construct
DIQMTQSPSSLSASVGDRVTITCRASQGISNYLAWFQQKPGKAPKSLIYAASSLQSGVPSKFSGSGSGTDFTLTISSLQSEDFATYYCQQYDSYPPTFGGGTKVEIKRTVAAPSVFI

SEQ ID NO: 19 - DNA, 351 bp, synthetic construct
  source 1..351: mol_type = other DNA
agctatgagctgacccagcctctgagcgtatctgtcgctctcggccagacagccagaattacctgtggcggcaataacataggatccaaaaatgttcactggtatcagcaaaaacctggccaagctcccgtgctcgtgatctaccgggactctaaccgacccagtggaatccccgaacgctttagcggttccaactctggaaatacagctactctgactatctccagggctcaggccggggatgaggccgattactactgccaggtgtgggactcaagcacagtggtcttcggcggaggtaccaagttgactgttcttgggcagccaaaggccgcaccttcagtgaccctg

SEQ ID NO: 20 - protein, 117 aa, synthetic construct
SYELTQPLSVSVALGQTARITCGGNNIGSKNVHWYQQKPGQAPVLVIYRDSNRPSGIPERFSGSNSGNTATLTISRAQAGDEADYYCQVWDSSTVVFGGGTKLTVLGQPKAAPSVTL

SEQ ID NO: 21 - protein, 5 aa, synthetic construct
SCVMT

SEQ ID NO: 22 - protein, 17 aa, synthetic construct
VISGSGGSTYYADSVKG

SEQ ID NO: 23 - protein, 12 aa, synthetic construct
TPWWLRSPFFDY

SEQ ID NO: 24 - protein, 11 aa, synthetic construct
GGNNIGSTSVH

SEQ ID NO: 25 - protein, 7 aa, synthetic construct
DDSDRPS

SEQ ID NO: 26 - protein, 10 aa, synthetic construct
VRDSSSDHVI

SEQ ID NO: 27 - protein, 5 aa, synthetic construct
FSYWV

SEQ ID NO: 28 - protein, 16 aa, synthetic construct
TIYYSGNTYYNPSLKS

SEQ ID NO: 29 - protein, 11 aa, synthetic construct
RAGILTGYLDS

SEQ ID NO: 30 - protein, 11 aa, synthetic construct
GGNNIGSKSVH

SEQ ID NO: 31 - protein, 7 aa, synthetic construct
DDSDRPS

SEQ ID NO: 32 - protein, 11 aa, synthetic construct
QVWDSSSDHVV

SEQ ID NO: 33 - protein, 5 aa, synthetic construct
GCYMH

SEQ ID NO: 34 - protein, 17 aa, synthetic construct
RINPNSGGTNYAQKFQG

SEQ ID NO: 35 - protein, 9 aa, synthetic construct
DYSGWSFDY

SEQ ID NO: 36 - protein, 11 aa, synthetic construct
RASQGISNYLA

SEQ ID NO: 37 - protein, 7 aa, synthetic construct
AASSLQS

SEQ ID NO: 38 - protein, 10 aa, synthetic construct
QQYDSYPPTF

SEQ ID NO: 39 - protein, 5 aa, synthetic construct
SYYWG

SEQ ID NO: 40 - protein, 16 aa, synthetic construct
NIYYSGSTYYNPSLMS

SEQ ID NO: 41 - protein, 13 aa, synthetic construct
QLSYNWNDRLFDY

SEQ ID NO: 42 - protein, 11 aa, synthetic construct
GGNNIGSKNVH

SEQ ID NO: 43 - protein, 7 aa, synthetic construct
RDSNRPS

SEQ ID NO: 44 - protein, 9 aa, synthetic construct
QVWDSSTVV

SEQ ID NO: 45 - DNA, 1350 bp, synthetic construct
  source 1..1350: mol_type = other DNA
cagctgctgctgcaagaatccggccctggcctggtgaaaccctccgagacactgtccctgacctgcaccgtgtccggcggctccctgtcctccagcttctcctactgggtctggatccggcagccccctggcaagggcctggaatggatcggcaccatctactactccggcaacacctactacaaccccagcctgaagtcccggctgaccatctccgtggacacctccaagaaccacttcagcctgaagctgtcctccgtgaccgccgctgacaccgccgtgtactactgtgccagaagggccggcatcctgaccggctacctggactcttggggccagggcaccctggtgacagtgtcctccgcctccaccaagggcccctccgtgttccctctggccccctccagcaagtccacctctggcggcaccgctgccctgggctgtctggtgaaagactacttccccgagcccgtgaccgtgtcctggaactctggcgccctgacctccggcgtgcacaccttccctgccgtgctgcagtcctccggcctgtactccctgtccagcgtggtgaccgtgccctccagctctctgggcacccagacctacatctgcaacgtgaaccacaagccctccaacaccaaggtggacaagcgggtggaacccaagtcctgcgacaagacccacacctgtcccccctgtcctgcccctgaagctgctggcggccctagcgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgcccccatcgaaaagaccatctccaaggccaagggccagccccgcgagccccaggtgtacacactgccccctagccgggaagagatgaccaagaaccaggtgtccctgacatgcctggtgaagggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggc

SEQ ID NO: 46 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - DNA, 642 bp, synthetic construct
  source 1..642: mol_type = other DNA
tcttacgtgctgacccagtccccctccgtgtccgtggctcctggccagaccgccagaatcacctgtggcggcaacaacatcggctccaagtccgtgcactggtatcagcagaagcccggccaggcccccgtgctggtggtgtacgacgactccgaccggccctctggcatccctgagcggttctccgcctccaactccggcaacaccgccaccctgaccatctccagagtggaagccggcgacgaggccgactactactgccaagtgtgggactcctcctccgaccacgtggtgttcggcggaggcaccaagctgaccgtgctgggccagcctaaggccgctccctccgtgaccctgttccccccatcctccgaggaactgcaggccaacaaggccaccctggtctgcctgatctccgacttctaccctggcgccgtgaccgtggcctggaaggccgacagctctcctgtgaaggccggcgtggaaaccaccaccccctccaagcagtccaacaacaaatacgccgcctcctcctacctgtccctgacccccgagcagtggaagtcccaccggtcctacagctgccaagtgacacacgagggctccaccgtggaaaagaccgtggcccctaccgagtgctcc

SEQ ID NO: 48 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - protein, 5 aa, synthetic construct
SSVMT

SEQ ID NO: 50 - protein, 11 aa, synthetic construct
GGSNIGSTSVH

SEQ ID NO: 51 - protein, 121 aa, synthetic construct
EVQLLESGGGLVQPGGSLRLSCAASEFTFGSSVMTWVRQAPGKGLEWVSVISGSGGSTYYADSVKGRFTISRDNSKNTLYLQMNSLRAEDTAVYYCAKTPWWLRSPFFDYWGQGTLVTVSS

SEQ ID NO: 52 - protein, 119 aa, synthetic construct
SYVLTQPPSVSVAPGQTARITCGGSNIGSTSVHWYQQKPGQAPVLVVHDDSDRPSGIPERFSGSNSGNTATLTISRVEAGDEADYYCQVRDSSSDHVIFGGGTKLTVLGQPKAAPSVTL

SEQ ID NO: 53 - protein, 450 aa, synthetic construct
EVQLLESGGGLVQPGGSLRLSCAASEFTFGSSVMTWVRQAPGKGLEWVSVISGSGGSTYYADSVKGRFTISRDNSKNTLYLQMNSLRAEDTAVYYCAKTPWWLRSPFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 214 aa, synthetic construct
SYVLTQPPSVSVAPGQTARITCGGSNIGSTSVHWYQQKPGQAPVLVVHDDSDRPSGIPERFSGSNSGNTATLTISRVEAGDEADYYCQVRDSSSDHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 55 - protein, 447 aa, synthetic construct
EVQLLESGGGLVQPGGSLRLSCAASEFTFGSSVMTWVRQAPGKGLEWVSVISGSGGSTYYADSVKGRFTISRDNSKNTLYLQMNSLRAEDTAVYYCAKTPWWLRSPFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 214 aa, synthetic construct; identical to SEQ ID NO: 54

SEQ ID NO: 57 - DNA, 1464 bp, synthetic construct
  source 1..1464: mol_type = other DNA
atgggctggtcctacatcatcctgtttctggtggccaccgccgccaccgtgcactccgaagtgcagctgctggaatccggcggaggcctggtgcagcctggcggctctctgagactgtcctgcgccgcctccgagttcaccttcggcagcagcgtgatgacctgggtccgacaggctcccggcaagggcctggaatgggtgtccgtgatctccggctccggcggctccacctactacgccgactctgtgaagggccggttcaccatctcccgggacaactccaagaacaccctgtacctgcagatgaactccctgcgggccgaggacaccgccgtgtactactgcgccaagaccccctggtggctgcggtcccccttcttcgattactggggccagggcaccctggtgacagtgtcctccgcctccaccaagggcccctccgtgttccctctggccccctccagcaagtccacctctggcggcaccgctgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgcaacgtgaaccacaagccctccaacaccaaggtggacaagcgggtggaacccaagtcctgcgacaagacccacacctgtcccccctgtcctgcccctgaagctgctggcggccctagcgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtctgtgctgaccgtgctgcaccaggactggctgaacggcaaagagtacaagtgcaaggtgtccaacaaggccctgcctgcccccatcgaaaagaccatctccaaggccaagggccagccccgcgagccccaggtgtacacactgccccctagccgggaagagatgaccaagaaccaggtgtccctgacctgtctggtgaaaggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggctgagtatacttaattaactgggcctcatgggccttccgctcactgcccgctttccag

SEQ ID NO: 58 - DNA, 702 bp, synthetic construct
  source 1..702: mol_type = other DNA
atgggctggtcctacatcatcctgtttctggtggccaccgccgccaccgtgcactcctcttacgtgctgacccagcccccctccgtgtctgtggctcctggccagaccgccagaatcacctgtggcggctcaaacatcggctccacctccgtgcactggtatcagcagaagcccggccaggcccccgtgctggtggtgcacgacgactccgaccggccttctggcatccctgagcggttctccggctccaactccggcaacaccgccaccctgaccatctccagagtggaagccggcgacgaggccgactactactgccaagtgcgagactcctcctccgaccacgtgatcttcggcggaggcaccaagctgaccgtgctgggccagcctaaggccgctccctccgtgaccctgttccccccatcctccgaggaactgcaggccaacaaggccaccctggtctgcctgatctccgacttctaccctggcgccgtgaccgtggcctggaaggccgacagctctcctgtgaaggccggcgtggaaaccaccaccccctccaagcagtccaacaacaaatacgccgcctcctcctacctgtccctgacccccgagcagtggaagtcccaccggtcctacagctgccaagtgacacacgagggctccaccgtggaaaagaccgtggcccctaccgagtgctcctga

SEQ ID NO: 59 - DNA, 1404 bp, synthetic construct
  source 1..1404: mol_type = other DNA
atgggctggtcctacatcatcctgtttctggtggctaccgccgccaccgtgcattctgaagttcagctgttggagtctggcggcggattggttcaacctggcggatctctgagactgtcttgcgccgcctctgagttcaccttcggctcctctgtgatgacctgggtccgacaggctcctggcaaaggactggaatgggtgtccgtgatctctggctctggcggctctacctactacgccgattctgtgaagggcagattcaccatcagccgggacaactccaagaacaccctgtacctgcagatgaactccctgagagccgaggacaccgccgtgtactattgtgctaagaccccttggtggctgcggtcccctttctttgattattggggccagggcaccctggtcaccgtgtcctctgcttctacaaagggcccctctgtgttccctctggctccttgctctagatccacctccgagtctaccgctgctctgggctgtctggtcaaggactactttcctgagcctgtgaccgtgtcttggaactctggtgctctgacctccggcgtgcacacatttccagctgtgctgcagtcctccggcctgtactctctgtcctctgtcgtgaccgtgccttctagctctctgggcaccaagacctacacctgtaacgtggaccacaagccttccaacaccaaggtggacaagcgcgtggaatctaagtacggccctccttgtcctccatgtcctgctccagaattcctcggcggaccctccgtgttcctgtttcctccaaagcctaaggacaccctgatgatctctcggacccctgaagtgacctgcgtggtggtggatgtgtctcaagaggaccccgaggtgcagttcaattggtacgtggacggcgtggaagtgcacaacgccaagaccaagcctagagaggaacagttcaactccacctacagagtggtgtctgtgctgaccgtgctgcaccaggattggctgaacggcaaagagtacaagtgcaaggtgtccaacaagggcctgccttccagcatcgaaaagaccatctccaaggctaagggccagcctcgggaacctcaggtttacaccctgcctccaagccaagaggaaatgaccaagaaccaggtgtccctgacctgcctcgtgaagggcttctacccttccgacattgccgtggaatgggagtctaacggccagccagagaacaactacaagacaacccctcctgtgctggactccgacggctctttcttcctgtattctcgcctgaccgtggacaagtctcggtggcaagagggcaacgtgttctcctgttctgtgatgcacgaggccctgcacaaccactacacccagaagtccctgtctctgtccctgggctaatga

SEQ ID NO: 60 - DNA, 702 bp, synthetic construct; identical to SEQ ID NO: 58
  source 1..702: mol_type = other DNA